(19) United States
(12) Patent Application Publication
(10) Pub. No.: US 2016/0000886 A1
(43) Pub. Date: Jan. 7, 2016

(54) NANOSTRUCTURED ACTIVE THERAPEUTIC VEHICLES AND USES THEREOF

(71) Applicant: PRESIDENT AND FELLOWS OF HARVARD COLLEGE, Cambridge, MA (US)

(72) Inventors: Kevin Kit Parker, Cambridge, MA (US); Johan Ulrik Lind, Boston, MA (US); David A. Weitz, Bolton, MA (US); Nichlaus J. Carroll, Belmont, MA (US); Alireza Abbaspourrad, Cambridge, MA (US)

(21) Appl. No.: 14/768,846

(22) PCT Filed: Feb. 21, 2014
(86) PCT No.: PCT/US14/17567

§ 371 Date: Aug. 19, 2015

Related U.S. Application Data
(60) Provisional application No. 61/768,206, filed on Feb. 22, 2013.

(51) Int. Cl. A61K 38/46; A61K 47/42; A61K 9/113; A61K 47/34; A61K 9/127; A61K 9/00

(52) U.S. Cl. CPC A61K 38/465; A61K 9/1273; A61K 9/0019; A61K 9/113; A61K 47/34; A61K 47/42; C12Y 301/01008

(57) ABSTRACT

The present invention provides nano structured active therapeutic vehicles which include a biodegradable polymeric fiber and/or thread comprising a porous particle which encapsulates an active agent. The vehicles of the present invention may be used to provide sustained release of the active agent to a subject.

### RELATED APPLICATIONS

This application claims priority to U.S. Provisional Application No. 61/768,206, filed Feb. 22, 2013, the entire contents of which are incorporated herein by reference.

### BACKGROUND OF THE INVENTION

In many instances, the effectiveness of an active agent, such as a therapeutically active agent, depends upon maintenance of a threshold concentration of the agent in vivo over prolonged time periods. To achieve continuous delivery of the active agent in vivo, a sustained release or sustained delivery vehicles or formulations are desirable, to avoid the need for repeated administrations.

Many extended-release vehicles and formulations which allow a two-fold or greater reduction in frequency of administration of an active agent in comparison with the frequency required by a conventional dosage form have been developed. These compositions are designed to deliver effective amounts of an active agent over extended periods of time following administration. This reduces labor costs by reducing the number of administration procedures during an overall treatment regimen. Extended release of the active agent also allows for treatment in situations where it would otherwise be impracticable. Further, effective extended release avoids large fluctuations in plasma levels of the active agent, initially too high and then rapidly too low, which occur upon injection of standard, non-extended release formulations.

However, for many therapeutically active agents, the preparation of extended release formulations and vehicles has failed due to the instability of the active agent. Furthermore, although conventional sealed extended-release vehicles and formulations, such as polymerosomes and liposomes, may increase the circulation time of an active agent, such vehicles do not permit immediate and on-demand availability of the active agent.

For example, in the case of butyrylcholinesterase (BuChE) which is a therapeutically active agent that provides short term protection against organophophorous nerve agents in various mammals (Lenz, D. E., et al. (2005) Chemico-Biological Interactions 157: 205-210; Lenz, D. E., et al. (2007) Toxicology 233(1-3): 31-39), in order to provide long term protection against nerve agents, the circulation time of the protein must be drastically increased. Extending and sustaining the circulation time should, at best, be done while still allowing the enzyme to bind nerve agents immediately upon exposure. Encapsulating BuChE in a conventional sealed polymerosome or liposome carrier could serve as a method for significantly extending the circulation time and furthermore facilitate oral administration of BuChE. However, such an approach requires detection of the nerve agent and release of the BuChE cargo prior to BuChE being capable of neutralizing the nerve agent. Additionally, prior to release, a threshold concentration of nerve agent is required as external triggering event.

Accordingly, there is a need in the art for improved vehicles that protect active agents encapsulated therein and extend and sustain the circulation time of the active agents.

### SUMMARY OF THE INVENTION

The present invention is based, at least in part, on the discovery of nanostructured active therapeutic vehicles which protect an active agent and extend the circulation time and, thus, availability of the active agent. In particular, it has been discovered that a vehicle comprising a biodegradable polymeric fiber and a biodegradable porous particle which encapsulates an active agent can provide extended and sustained release of the active agent. The porous particle is selectively permeable and, in some embodiments, the porous particle concurrently allows free passage of, e.g., a toxin, into the porous particle while inhibiting diffusion of, e.g., a protein, such as a protease, into the porous particle. The selective porosity of the particles takes advantage of the size differences in, e.g., toxins which are typicaly less than about 300 Daltons, and proteins, such as proteases which are typically greater than about 10 kDaltons. This selective porosity is useful in, for example, preventing degradation of the active agent encapsulated within the porous particle when the vehicle is administered to a subject. The biodegradable polymer acts as a depot providing continuous release of the porous particles extending and sustaining circulation time of the porous particles and, thus, the active agent.

Accordingly, the present invention provides sustained release compositions and methods of use thereof.

In one aspect, the present invention provides nano structured active therapeutic vehicles. The vehicles include a biodegradable polymeric fiber comprising a porous particle, wherein the porous particle comprises regulators that control passage of molecules into and out of the particle, and wherein the porous particle comprises an active agent.

In another aspect, the present invention provides nanostructured active therapeutic vehicles for sustained delivery of an active agent. The vehicles include a biodegradable polymeric fiber and a polymerosome comprising the active agent, wherein the active agent is an agent which inhibits the activity of a toxin, and wherein the polymerosome comprises size regulators which control passage of molecules into and out of the particle such that the active agent is excluded from exiting the polymerosome, a molecule which degrades the active agent is excluded from entry into the polymerosome, and the toxin is permitted entry into the polymerosome such that the toxin contacts the active agent, thereby inhibiting the activity of the toxin.

In one aspect, the present invention provides nano structured active therapeutic vehicles which include a biodegradable polymeric thread comprising a porous particle, wherein the porous particle comprises regulators that control passage of molecules into and out of the particle, and wherein the porous particle comprises an active agent.

In another aspect, the present invention provides nanostructured active therapeutic vehicle for sustained delivery of an active agent which include a biodegradable polymeric thread and a polymerosome comprising the active agent, wherein the active agent is an agent which inhibits the activity of a toxin, and wherein the polymerosome comprises size regulators which control passage of molecules into and out of the particle such that the active agent is excluded from exiting the polymerosome, a molecule which degrades the active agent is excluded from entry into the polymerosome, and the toxin is permitted entry into the polymerosome such that the toxin contacts the active agent, thereby inhibiting the activity of the toxin.

In one aspect, the present invention provides methods for providing sustained release of an active agent to a subject having a condition treatable with the active agent. The methods include administering to the subject an effective amount of a nanostructured active therapeutic vehicle comprising the active agent, wherein the nanostructured active therapeutic vehicle comprises a biodegradable polymeric fiber comprising a porous particle, wherein the porous particle comprises regulators that control passage of molecules into and out of the particle, and wherein the porous particle comprises an active agent, thereby providing sustained release of the active agent to the subject having a condition treatable with the active agent.

In another aspect, the present invention provides methods for providing sustained release of an active agent which inhibits the activity of a toxin in a subject, such as a subject at risk of being exposed to the toxin. The methods include administering to the subject an effective amount of nano structured active therapeutic vehicle comprising an active agent that inhibits the activity of the toxin, wherein the nanostructured active therapeutic vehicle comprises a biodegradable polymeric fiber comprising a polymerosome, and wherein the polymerosome comprises size regulators which control passage of molecules into and out of the particle such that the active agent is excluded from exiting the polymerosome, a molecule which degrades the active agent is excluded from entry into the polymerosome, and the toxin is permitted entry into the polymerosome such that the toxin contacts the active agent, thereby providing sustained release of an active agent which inhibits the activity of a toxin to the subject.

In yet another aspect, the present invention provides method for inhibiting the activity of a toxin in a cell. The methods include comprising contacting the cell with nanostructured active therapeutic vehicle comprising an active agent capable of inhibiting the activity of the toxin, wherein the nanostructured active therapeutic vehicle comprises a biodegradable polymeric fiber comprising a porous particle, wherein the porous particle comprises regulators that control passage of molecules into and out of the particle, and wherein the porous particle comprises an active agent, thereby inhibiting the activity of a toxin in the cell.

In one aspect, the present invention provides methods for providing sustained release of an active agent to a subject having a condition treatable with the active agent. The methods include administering to the subject an effective amount of a nanostructured active therapeutic vehicle comprising the active agent, wherein the nanostructured active therapeutic vehicle comprises a biodegradable polymeric thread comprising a porous particle, wherein the porous particle comprises regulators that control passage of molecules into and out of the particle, and wherein the porous particle comprises an active agent, thereby providing sustained release of the active agent to the subject having a condition treatable with the active agent.

In another aspect, the present invention provides methods for providing sustained release of an active agent which inhibits the activity of a toxin in a subject, such as a subject at risk of being exposed to the toxin. The methods included administering to the subject an effective amount of nano structured active therapeutic vehicle comprising an active agent that inhibits the activity of the toxin, wherein the nanostructured active therapeutic vehicle comprises a biodegradable polymeric thread comprising a polymerosome, and wherein the polymerosome comprises size regulators which control passage of molecules into and out of the particle such that the active agent is excluded from exiting the polymerosome, a molecule which degrades the active agent is excluded from entry into the polymerosome, and the toxin is permitted entry into the polymerosome such that the toxin contacts the active agent, thereby providing sustained release of an active agent which inhibits the activity of a toxin to the subject.

In yet another aspect, the present invention provides methods for inhibiting the activity of a toxin in a cell. The methods include contacting the cell with nanostructured active therapeutic vehicle comprising an active agent capable of inhibiting the activity of the toxin, wherein the nanostructured active therapeutic vehicle comprises a biodegradable polymeric thread comprising a porous particle, and wherein the porous particle comprises the active agent, thereby inhibiting the activity of a toxin in the cell.

The nanostructured active therapeutic vehicle comprising an active agent may be administered to the subject subcutaneously, such as subcutaneous suturing.

The biodegradable polymeric fiber and/or thread may comprise a synthetic polymer, such as poly(urethanes), poly(siloxanes) or silicones, poly(ethylene), poly(vinyl pyrrolidone), poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactides (PLA), polyglycolides (PGA), poly(lactide-co-glycolides) (PLGA), poly(dioxanone), polyanhydrides, polyphosphazenes, polygermanes, polyorthoesters, polyesters, polyamides, polyolefins, polycarbonates, polyaramides, polyimides, and copolymers and derivatives thereof, and/or a natural polymer, such as silk, keratins, fibrillins, fibrinogen, fibrins, thrombin, fibronectin, laminin, collagens, vimentin, neurofilaments, amyloids, actin, myosins, titin, chitin, hyaluronic acid, glycosaminoglycans, gelatin, albumin, and combinations thereof.

The polymeric fiber and/or thread may be about 1 to about 1,000, 1-900, 1-800, 1-700, 1-600, 1-500, 1-400, 1-300, 1-200, 1-100, 5-1,000, 5-900, 5-800, 8-700, 5-600, 5-500, 5-400, 5-300, 5-200, 5-100, 5-50, 10-1,000, 10-900, 10-800, 10-700, 10-600, 10-500, 10-400, 10-300, 10-200, 10-100, or about 10 to about 50 micrometers in diameter, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 55, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 500, 550, 600, 650, 700, 750, 800, 850, 900, 950, or 1,000 micrometers in diameter.

The tensile strength of the polymeric fiber and/or thread may be about 0.5 N to about 100 N, or about 1 N to about 50 N, or about 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 N.

The porous particle may be an emulsion product, e.g., a polymerosome, a liposome, a microcapsule, or a nanocapsule, a microgel or a particle whose pores may be templated by micelles, microemulsion drops, dendrimers, colloids, liquid porogen, lipids, degree of polymeric crosslinks, a dendrimer, a micelle or any combination thereof.

The polymerosome may have a diameter of about 0.1 to about 10 micrometers, or about 0.5 to about 5 micrometers, or about 01, 0.2, 0.3, 0.4, 0.5, 0.6, 0.7, 0.8, 0.9, 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, or about 10 micrometers.

The polymerosome may have a shell with a thickness of about 50 to about 500 nanometers, or about 50, 51, 52, 53, 55, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, or about 500 nanometers.

The polymerosome may be impermeable to molecules greater than about 10 kiloDaltons, but permeable to molecules about 5 to about 500 Daltons, or about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 55, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, or about 500 Daltons.

The polymerosome may have a stiffness of about 5 to about 100 kiloPascals, or about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 55, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 kiloPascals.

The middle layer of the polymerosome may be a polymer such as poly(ε-caprolactone), PLA, PLGA, PHB, POE, PHBV, copolymers, and/or derivatives thereof.

The outer layer of the polymerosome may comprise polyethylene glycol or CD47.

The active agent may be small molecules, nucleic acid based drugs; polypeptides; peptides; proteins; carbohydrates; polysaccharides and other sugars; glycoproteins, and/or lipids. In one embodiment, the active agent is butyrlcholinesterase.

The nanostructured active therapeutic vehicle may provide release of the active agent for about 1 week to about 1 month, or about 1 week to about 3 months.

### BRIEF DESCRIPTION OF THE DRAWINGS

FIGS. 1A-1C depict one embodiment of the nanostructured active therapeutic vehicles of the invention to provide long term protection against nerve agents A) A polymerosome with nanopores that allows fast entry of small molecule nerve agents, while preventing larger proteins from crossing the membrane. B) An administration system, based on subcutaneously suturing a biodegradable fiber and/or thread which upon degradation, slowly releases the polymerosomes. C) The spatial scale of the delivery system.

FIGS. 2A-2C depict an embodiment of devices and methods for the fabrication of double-emulsions. A) Schematic illustration of a microfluidic device for preparation of double-emulsion drops with an ultra-thin shell. B) Double emulsion drops produced within a glass capillary device. C) Optical micrograph of resultant double emulsions.

FIGS. 3A-3I depict an exemplary Rotary Jet Spinning (RJS) device and use thereof for the fabrication of polymeric fibers and/or threads, as well as exemplary fibers fabricated using such devices and methods. A) Schematic of one embodiment of a rotary jet spinning device used to fabricate biodegradable fibers and/or threads encapsulating polymerosomes. B-G) Fibers formed using RJS B-C: PLA, D: Gelatin co-spun with PLA, E: PEG, F: PAA, G: PEG fibers encapsulating 200 nm fluorescently labeled polystyrene beads. H-I) PLA microfiber suture.

FIG. 4 depicts an exemplary embodiment of in-situ photo-polymerization of template double droplets to form capsules with porous membrane and functionalized surface.

FIG. 5 is a schematic of an in vitro fluorescence permeability assay.

FIGS. 6A and 6B depict the biodegradation of polymeric fibers and threads. A) Schematic of in vitro biodegradation assay with fibroblasts cultured with fibers and/or threads in a transwell plate. B) Alteration in mass of a fiber mesh cultured with cardiac fibroblasts after 4 weeks in transwell culture (N=9 samples, * indicate p<0.05; box plot: 25-75%, error bars: 10-90%).

FIG. 7A depicts an exemplary embossing tool fabricated in silicon (Becker, et al. (2000)).

FIG. 7B is the chemical structure of fluorinated ethylene propylene (FEP).

FIG. 7C depicts a nickel master, resultant FEP device, and a schematic of a hot embossing technique.

FIGS. 8A and 8B are optical micrographs of deformed capsules conforming locally to a force-calibrated microcantilever tip.

FIG. 8C depicts the deformation of an unpressurized thin elastic shell.

FIG. 9 depicts an exemplary in vivo analysis of biodegradable fibers and/or threads for use in the nanostructured active therapeutic vehicles of the present invention. A) Fibers are introduced on the dorsal side of the mouse. B) Diameter and weight of collected fibers is used to estimate fiber degradation. C) Histology identifies potential immune response, fiber degradation and local microparticle distribution. D) Blood collected from sacrificed mice verifies the presence of N-IR fluorescently labeled microparticles. E) Infrared scanning of whole mice is used to investigate the in vivo distribution and aggregation of labeled porous particles. F) Intravital microscopy verifies the in vivo circulation of IV-injected N-IR labeled porous particles.

FIG. 10A is a schematic of a microfluidic filter.

FIGS. 10B and 10C are optical micrographs of the B) inlet and C) outlet of the microfluidic filter.

FIG. 10D is a schematic of a microfluidic filter where the emulsion is formed on-chip.

FIG. 10E depicts double emulsions split into smaller drops using splitting junctions.

FIGS. 11A-11C depict an in vitro assay of activity of polymerosome activity against nerve agents. A) Ellman assay of AChE activity. AChE hydrolyzes ATCh to form TCh. TCh reacts with DTNB to form TNB which has a strong absorbance at 412 nm. B) When a nerve agents binds to AChE it becomes inactive, fails to hydrolyze ATCh, and there is no increase in absorbance at 412 nm. C) The ability of BuChE filled polymerosomes to capture nerve agents is assessed by exposing AChE to nerve agents in the presence of polymerosomes, and performing an Ellman assay. The polymerosomes are filtered off via dialysis prior to the fluorescence assay if the encapsulated BuChE contributes to the hydrolysis of the applied ATCh.

### DETAILED DESCRIPTION OF THE INVENTION

The present invention provides sustained release compositions and methods of use thereof.

### I. Nanostructured Active Therapeutic Vehicles

As used herein, the term “nanostructured active therapeutic vehicle”, is a composition which provides extended and sustained release of an active agent. A nanostructured active therapeutic vehicle comprises a biodegradable polymeric fiber and/or thread and a porous particle, e.g., a biodegradable porous particle, wherein the porous particle comprises an active agent. Nanostructured active therapeutic vehicles may be fabricated by contacting a biodegradable polymeric fiber and/or thread with a porous particle, e.g., a biodegradable porous particle, encapsulating a therapeutically active agent. Porous particles, polymeric fibers and/or threads, and therapeutically active agents suitable for use in the compositions and methods of the invention, as well as methods of fabricating biodegradable porous particles encapsulating an active agent and biodegradable polymeric fibers and/or threads are described in the subsections below.

### A. Porous Particles

Suitable porous particles comprising an active agent for use in the nanostructured active therapeutic vehicles of the present invention, include, for example, emulsion products (such as polymerosomes, liposomes, colloidosomes, micro- and nanocapsules, microgels and particles whose pores can be templated by micelles, microemulsion drops, dendrimers, colloids, liquid porogen, lipids, degree of polymeric crosslinks or any combination thereof.

The pores of the porous particle selectively regulate passage of molecules into and out of the particle and are referred to herein as “regulators.” A regulator controls the passage of molecules into and out of the particle based on differences in, for example, size, hydrophobicity, and/or charge of molecules. For example, a regulator which controls the passage of molecules based on size may permit entry of molecules that are about 5 to about 500 Daltons (about 5-450, 5-400, 5-300, 10-500, 10-400, or about 10-300 Daltons, or about 5, 10, 25, 50, 75, 100, 125, 150, 175, 200, 225, 250, 275, 300, 325, 350, 375, 400, 425, 450, 475, or about 500 Daltons) into the porous particle while excluding molecules greater than about 10 kDaltons (about 5-150, 5-100, 10-150, or about 10-100 kDaltons, or about 10, 20, 30, 40, 50, 60, 70, 80, 90, 100, 110, 120, 130, 140, or about 150 kDaltons) from the particle.

The porous particles for use in the nanostructured active therapeutic vehicles of the present invention typically have a mean diameter of from about 1-200 μm, 1-100 μm, 1-80 μm, 1-50 μm, 1-30 μm, 20-40 μm, 1-10 μm, or 1-5 μm, or a mean diameter of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100 μm. Ranges and values intermediate to the above recited ranges and values are also contemplated to be part of the invention.

In one embodiment, the porous particle to active agent ratio (mass/mass ratio) (e.g., polymer to active agent ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1. Ranges intermediate to the above recited ranges are also contemplated to be part of the invention.

Porous particles and methods and devices for making the porous particles have been described in U.S. Pat. No. 7,776,927 and U.S. Patent Application Publication No. 20130046030, 20120222748, 20120211084, 20120199226, 20120141589, 20120107601 10 20120015822, 20120015382, 20110275063, 20110229545, 20110218123, 20110190146, 20110123413, 20100213628, 20100172803, 20090012187, all of which are hereby incorporated by reference in their entirety.

1. Emulsions, Multiple Emulsions, and Emulsion Products

In one embodiment, a porous particle comprising an active agent for use in the nanostructured active therapeutic vehicles of the present invention is an emulsion and/or a multiple emulsion product.

An “emulsion” is a fluidic state which exists when a first fluid is dispersed in the form of droplets in a second fluid that is typically immiscible or substantially immiscible with the first fluid. Examples of common emulsions are oil in water and water in oil emulsions.

“Multiple emulsions” are emulsions that are formed with more than two fluids, or two or more fluids arranged in a more complex manner than a typical two-fluid emulsion. For example, a multiple emulsion may be oil-in-water-in-oil (O/W/O), or water-in-oil-in-water (W/O/W).

A multiple emulsion typically comprises larger droplets that contain one or more smaller droplets therein. The larger droplet or droplets may be suspended in a third fluid in some cases. In certain embodiments, emulsion degrees of nesting within the multiple emulsion are possible. For example, an emulsion may contain droplets containing smaller droplets therein, where at least some of the smaller droplets contain even smaller droplets therein, etc. In some cases, one or more of the droplets (e.g., an inner droplet and/or an outer droplet) can change form, for instance, to become solidified to form a microcapsule, a liposome, a polymerosome, or a colloidosome.

As described below, multiple emulsions can be formed in one step in certain embodiments, with generally precise repeatability, and can be tailored to include one, two, three, or more inner droplets within a single outer droplet (which droplets may all be nested in some cases). As used herein, the term “fluid” generally means a material in a liquid or gaseous state. Fluids, however, may also contain solids, such as suspended or colloidal particles.

Typically, however, multiple emulsions consisting of a droplet inside another droplet are made using a two-stage emulsification technique, such as by applying shear forces through mixing to reduce the size of droplets formed during the emulsification process. Other methods such as membrane emulsification techniques using, for example, a porous glass membrane, have also been used to produce water-in-oil-in-water emulsions. Microfluidic techniques have also been used to produce droplets inside of droplets using a procedure including two or more steps. For example, see International Patent Application No. PCT/US2004/010903, filed Apr. 9, 2004, entitled “Formation and Control of Fluidic Species,” by Link, et al., published as WO 2004/091763 on Oct. 28, 2004; or International Patent Application No. PCT/US03/20542, filed Jun. 30, 2003, entitled “Method and Apparatus for Fluid Dispersion,” by Stone, et al., published as WO 2004/002627 on Jan. 8, 2004, each of which is incorporated herein by reference. See also Anna, et al., “Formation of Dispersions using ‘Flow Focusing’ in Microchannels,” Appl. Phys. Lett., 82:364 (2003) and Okushima, et al., “Controlled Production of Monodispersed Emulsions by Two-Step Droplet Breakup in Microfluidic Devices,” Langmuir 20:9905-9908 (2004). In some of these examples, a T-shaped junction in a microfluidic device is used to first form an aqueous droplet in an oil phase, which is then carried downstream to another T-junction where the aqueous droplet contained in the oil phase is introduced into another aqueous phase. In another technique, co-axial jets can be used to produce coated droplets, but these coated droplets must be re-emulsified into the continuous phase in order to form a multiple emulsion. See Loscertales et al., “Micro/Nano Encapsulation via Electrified Coaxial Liquid Jets,” Science 295:1695 (2002).

In one aspect, the multiple emulsions described herein may be made in a single step using different fluids. In one set of embodiments, a triple emulsion may be produced, i.e., an emulsion containing a first fluid, surrounded by a second fluid, which in turn is surrounded by a third fluid. In some cases, the third fluid and the first fluid may be the same. These fluids can be referred to as an inner fluid (IF), a middle fluid (MF) and an outer fluid (OF), respectively, and are often of varying miscibilities due to differences in hydrophobicity. For example, the inner fluid may be water soluble, the middle fluid oil soluble, and the outer fluid water soluble. This arrangement is often referred to as a w/o/w multiple emulsion (“water/oil/water”). Another multiple emulsion may include an inner fluid that is oil soluble, a middle fluid that is water soluble, and an outer fluid that is oil soluble. This type of multiple emulsion is often referred to as an o/w/o multiple emulsion (“oil/water/oil”). It should be noted that the term “oil” in the above terminology merely refers to a fluid that is generally more hydrophobic and not miscible in water, as is known in the art. Thus, the oil may be a hydrocarbon in some embodiments, but in other embodiments, the oil may comprise other hydrophobic fluids.

As used herein, two fluids are immiscible, or not miscible, with each other when one is not soluble in the other to a level of at least 10% by weight at the temperature and under the conditions at which the multiple emulsion is produced. For instance, the fluid and the liquid may be selected to be immiscible within the time frame of the formation of the fluidic droplets. In some embodiments, the inner and outer fluids are compatible, or miscible, while the middle fluid is incompatible or immiscible with each of the inner and outer fluids. In other embodiments, however, all three fluids may be mutually immiscible, and in certain cases, all of the fluids do not all necessarily have to be water soluble. In still other embodiments, additional fourth, fifth, sixth, etc. fluids may be added to produce increasingly complex droplets within droplets, e.g., a first fluid may be surrounded by a second fluid, which may in turn be surrounded by a third fluid, which in turn may be surrounded by a fourth fluid, etc.

In the descriptions herein, multiple emulsions are generally described with reference to a three phase system, i.e., having an outer fluid, a middle fluid, and an inner fluid. However, it should be noted that this is by way of example only, and that in other systems, additional fluids may be present within the multiple droplet. As examples, an emulsion may contain a first fluid droplet and a second fluid droplet, each surrounded by a third fluid, which is in turn surrounded by a fourth fluid; or an emulsion may contain multiple emulsions with higher degrees of nesting. Accordingly, it should be understood that the descriptions of the inner fluid, middle fluid, and outer fluid are by ways of ease of presentation, and that the descriptions below are readily extendable to systems involving additional fluids.

As fluid viscosity can affect droplet formation, in some cases the viscosity of the inner, middle, and/or outer fluids may be adjusted by adding or removing components, such as diluents, that can aid in adjusting viscosity. In some embodiments, the viscosity of the inner fluid and the middle fluid are equal or substantially equal. This may aid in, for example, an equivalent frequency or rate of droplet formation in the inner and middle fluids. In other embodiments, the outer fluid may exhibit a viscosity that is substantially different from either the inner or middle fluids. A substantial difference in viscosity means that the difference in viscosity between the two fluids can be measured on a statistically significant basis. Other distributions of fluid viscosities within the droplets are also possible. For example, the inner fluid may have a viscosity greater than or less than the viscosity of the middle fluid, the middle fluid may have a viscosity that is greater than or less than the viscosity of the outer fluid, etc. It should also be noted that, in higher-order droplets, e.g., containing four, five, six, or more fluids, the viscosities may also be independently selected as desired, depending on the particular application.

Emulsions can contain additional components in addition to the dispersed phases, and an active agent which can be present as a solution in either the aqueous phase, oily phase or itself as a separate phase. Pharmaceutical excipients such as emulsifiers, stabilizers, dyes, and anti-oxidants can also be present in emulsions as needed. Pharmaceutical emulsions can also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous phase provides an o/w/o emulsion.

Synthetic surfactants, also known as surface active agents, have found wide applicability in the formulation of emulsions and have been reviewed in the literature (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N. Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants can be classified into different classes based on the nature of the hydrophilic group: nonionic, anionic, cationic and amphoteric (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y. Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that can readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used can be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

Porous particles comprising a hardened shell, such as polymersomes, liposomes, colloidosomes, micro- and nano-capsules (polymerosomes comprise a single bi-layer of polymer, capsules comprise shells with thickness of tens of nanometers up to microns and are not limited to bilayers) are prepared from emulsions. In one embodiment, a hardened shell may be formed around an inner droplet, such as by using a middle fluid that can be solidified or gelled. In one embodiment, this can be accomplished by a phase change in the middle fluid. A “phase change” fluid is a fluid that can change phases, e.g., from a liquid to a solid. A phase change can be initiated by a temperature change, for instance, and in some cases the phase change is reversible. For example, a wax or gel may be used as a middle fluid at a temperature which maintains the wax or gel as a fluid. Upon cooling, the wax or gel can form a solid or semisolid shell, e.g., resulting in a capsule. The shell may also be a bilayer, such as a shell formed from two layers of surfactant. Exemplary porous particles comprising hardened shells are described below.

In one embodiment, multiple emulsions are formed by flowing three (or more) fluids through a system of conduits. The system may be a microfluidic system. “Microfluidic,” as used herein, refers to a device, apparatus or system including at least one fluid channel having a cross-sectional dimension of less than about 1 millimeter (mm), and in some cases, a ratio of length to largest cross-sectional dimension of at least 3:1. One or more conduits of the system may be a capillary tube. In some cases, multiple conduits are provided, and in some embodiments, at least some are nested, as described herein. The conduits may be in the microfluidic size range and may have, for example, average inner diameters, or portions having an inner diameter, of less than about 1 millimeter, less than about 300 micrometers, less than about 100 micrometers, less than about 30 micrometers, less than about 10 micrometers, less than about 3 micrometers, or less than about 1 micrometer, thereby providing droplets having comparable average diameters. One or more of the conduits may (but not necessarily), in cross section, have a height that is substantially the same as a width at the same point. Conduits may include an orifice that may be smaller, larger, or the same size as the average diameter of the conduit. For example, conduit orifices may have diameters of less than about 1 mm, less than about 500 micrometers, less than about 300 micrometers, less than about 200 micrometers, less than about 100 micrometers, less than about 50 micrometers, less than about 30 micrometers, less than about 20 micrometers, less than about 10 micrometers, less than about 3 micrometers, etc. In cross-section, the conduits may be rectangular or substantially non-rectangular, such as circular or elliptical. The conduits of the present invention can also be disposed in or nested in another conduit, and multiple nestings are possible in some cases. In some embodiments, one conduit can be concentrically retained in another conduit and the two conduits are considered to be concentric. In other embodiments, however, one conduit may be off-center with respect to another, surrounding conduit. By using a concentric or nesting geometry, the inner and outer fluids, which are typically miscible, may avoid contact facilitating great flexibility in making multiple emulsions and in devising techniques for encapsulation and polymerosome formation. For example, this technique allows for fabrication of core-shell structure, and these core-shell structures can be converted into capsules.

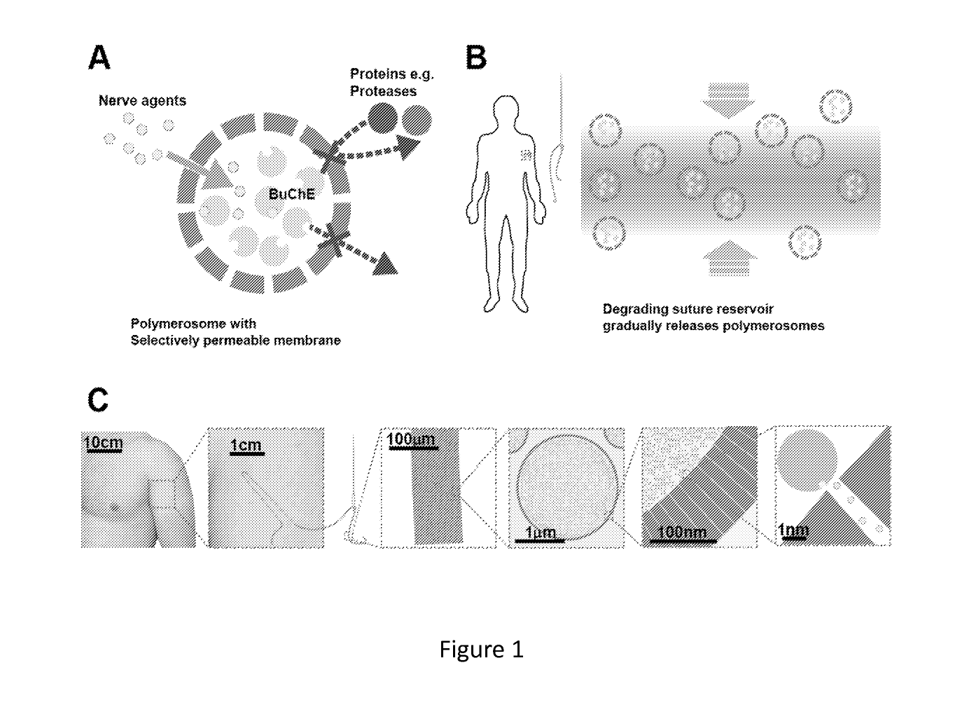

In one embodiment, the emulsions are prepared using a capillary microfluidic device comprised of a hydrophobic tapered injection capillary inserted into a second square capillary (made from, for example, AIT glass) whose inner dimension is the same as that of the outer diameter of the injection capillary, which is, for example, 1 mm, as schematically illustrated in FIG. 1a. In an embodiment, the capillary wall is made hydrophobic using, for example, n-octadecyltrimethoxy silane. In addition, a small tapered capillary is inserted into the injection capillary to simultaneously inject a second immiscible fluid, as shown in FIG. 1a. Another circular capillary is inserted into the square capillary at the other side to confine the flow near the injection tip, thereby increasing the flow velocity. The circular capillary wall is made hydrophilic by coating with, for example, 2-[methoxy(polyethyleneoxy)propyl]trimethoxy silane. In an embodiment, an aqeous solution of, for example, PEG, is injected through the small tapered capillary as the inner fluid to form the inner drops; a solvent solution of, for example, hexadecane with SPAN 80 is injected through the injection capillary as the middle fluid; and an aqeous solution of, for example, poly(vinyl alcohol), is injected through the square capillary as the outer fluid.

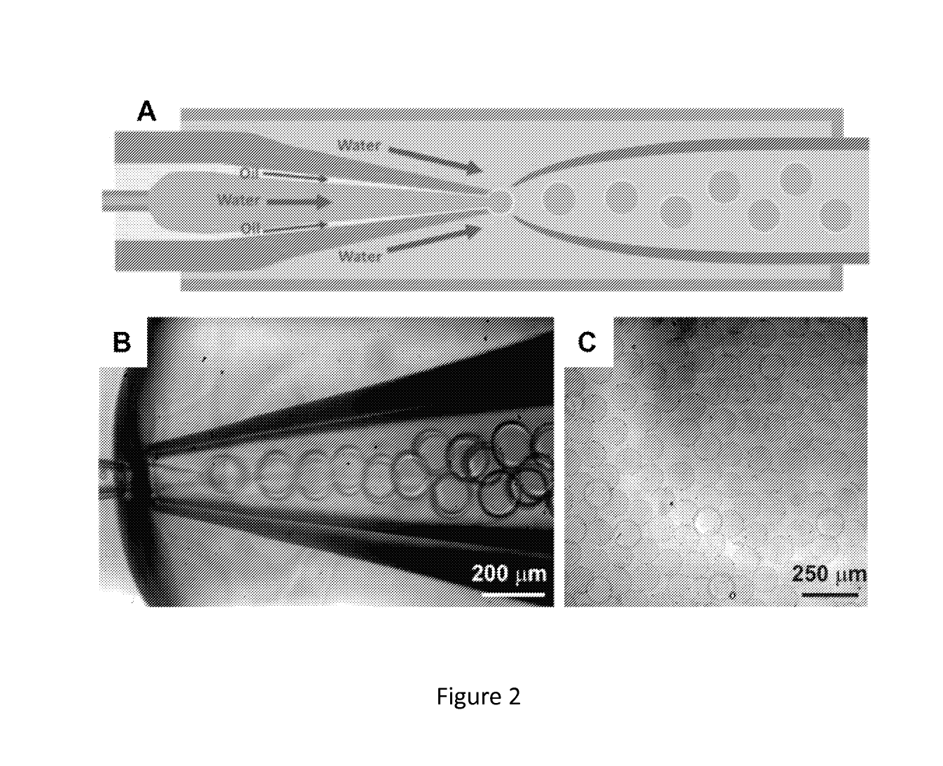

In one embodiment, monodisperse double-emulsion drops with an ultra-thin middle layer is prepared by using a single-step emulsification in a capillary microfluidic device. In this approach, highly monodisperse double emulsion drops are generated and subsequently converted into robust core-shell capsules, by consolidation of the ultra-thin middle layer (FIG. 2A). A biphasic flow is created, consisting of a sheath of one fluid flowing along the capillary wall and surrounding a second fluid flowing through the center of the capillary. Two immiscible fluids which flow coaxially and simultaneously through a single capillary can exhibit two distinct flow patterns, consisting of either a coaxial jet or a stream of drops of one fluid in the second. A jet of one liquid in the second is typically unstable to the Rayleigh-Plateau instability which causes a breakup of the jet into drops; this instability can be suppressed by confining the coaxial flow. Further control over the fluid flow can be achieved by exploiting the affinity of the fluid to the capillary; the fluid with higher affinity to the wall will flow along it whereas the second fluid will flow through the center of the capillary. Because of the affinity to the wall, the thickness of the outer fluid can be very thin. By controlling the thickness of the fluid with high affinity to the wall, double-emulsion drops with an ultra-thin middle layer can be produced using a one-step emulsification process. The thickness can also be tuned by adjusting the relative flow rate of the fluids, the polymer/solvent ratio or by exploiting a co-flowing biphasic flow capillary geometry to form ultra-thin shells. The foregoing method can be used to form shells with thicknesses of 100 nm or less, which will facilitate the fast diffusion of toxins into the capsule core. This biphasic flow forms double-emulsion drops that have core-shell structure with a very thin outer wall. This technique enables the preparation of double-emulsion drops with highly viscous organic solvents, facilitating the formation of functional microcapsules with an ultra-thin membrane. Biodegradable microcapsules with a shell thickness of a few tens of nanometers using evaporation-induced solidification in water-in-oil-in-water (W/O/W) double-emulsion drops.

A variety of materials and methods can be used to form any of the above-described components of the devices. In some cases, the various materials selected lend themselves to various methods. For example, various components of the devices can be formed from solid materials, in which the channels can be formed via micromachining, film deposition processes such as spin coating and chemical vapor deposition, laser fabrication, photolithographic techniques, etching methods including wet chemical or plasma processes, and the like. In one embodiment, at least a portion of the fluidic system is formed of silicon by etching features in a silicon chip. Technologies for precise and efficient fabrication of various fluidic systems and devices of the invention from silicon are known. In another embodiment, various components of the systems and devices of the invention can be formed of a polymer, for example, an elastomeric polymer such as polydimethylsiloxane (“PDMS”), polytetrafluoroethylene (“PTFE”), or the like.

Different components can be fabricated of different materials. For example, a base portion including a bottom wall and side walls can be fabricated from an opaque material such as silicon or PDMS, and a top portion can be fabricated from a transparent or at least partially transparent material, such as glass or a transparent polymer, for observation and/or control of the fluidic process. Components can be coated so as to expose a desired chemical functionality to fluids that contact interior channel walls, where the base supporting material does not have a precise, desired functionality. For example, components can be fabricated as illustrated, with interior channel walls coated with another material. Material used to fabricate various components of the systems and devices of the invention, e.g., materials used to coat interior walls of fluid channels, may desirably be selected from among those materials that will not adversely affect or be affected by fluid flowing through the fluidic system, e.g., material(s) that is chemically inert in the presence of fluids to be used within the device.

a. Polymersomes

When an amphiphilic polymer, such as a diblock copolymer, is used as the majority component in an emulsion, the resulting droplets with a hardened shell can be referred to as polymerosomes (polymer vesicles). In one embodiment, polymersomes are formed when the middle fluid droplet of a multiple emulsion is solidified to form a shell. The solidification of the drop middle phase can be performed using solvent evaporation, polymerization, or dewetting of the middle phase onto the surface of the innermost drop.

Solvent evaporation initiates dewetting to form polymerosomes consisting of a bilayer of amphiphilic polymer. However, solvent evaporation of a middle phase containing non-amphiphilic linear polymer will result in a consolidated polymeric shell much thicker than just a single bilayer to form a capsule.

Polymersomes can be spherical or non-spherical. They can also have a single compartment or have multiple compartments. The properties of polymersomes, such as polymer length, biocompatibility, functionality, and degradation rates, spherical polymersomes with a single compartment, nonspherical polymersomes with multiple compartments can be tailored for specific active agents. Synthasomes are polymersomes engineered to contain channels (formed using for example, transmembrane proteins or other pore-forming molecules) that allow certain chemicals to pass through the membrane, into or out of the vesicle.

In one embodiment, polymerization to form the polymersome shell can be accomplished using various methods, including using a pre-polymer that can be catalyzed, for example, chemically, through heat, or via electromagnetic radiation (e.g., ultraviolet radiation) to form a solid polymer shell. Polymers may include polymeric compounds, as well as compounds and species that can form polymeric compounds, such as prepolymers. Prepolymers include, for example, monomers and oligomers. In some cases, however, only polymeric compounds are used and prepolymers may not be appropriate. The polymersomes can also be made from “block copolymer.” Block copolymers are polymers having at least two, tandem, interconnected regions of differing chemistry. Each region comprises a repeating sequence of monomers. Thus, a “diblock copolymer” comprises two such connected regions (A-B); a “triblock copolymer,” three (A-B-C), etc. Each region may have its own chemical identity and preferences for solvent.

Multiple emulsions can be formed that include amphiphilic species such as amphiphilic polymers and lipids and amphiphilic species typically includes a relatively hydrophilic portion, and a relatively hydrophobic portion. For instance, the hydrophilic portion may be a portion of the molecule that is charged, and the hydrophobic portion of the molecule may be a portion of the molecule that comprises hydrocarbon chains. The polymerosomes may be formed, for example, in devices such as those described above with respect to multiple emulsions. As mentioned above, one or more of the fluids forming the multiple emulsions may include polymers, such as copolymers, which can be subsequently polymerized. An example of such a system is normal butyl acrylate and acrylic acid, which can be polymerized to form a copolymer of poly(normal-butyl acrylate)-poly(acrylic acid).

In some cases, upon formation of a multiple emulsion, an amphiphilic species that is contained, dissolved, or suspended in the emulsion can spontaneously associate along a hydrophilic/hydrophobic interface in some cases. For instance, the hydrophilic portion of an amphiphilic species may extend into the aqueous phase and the hydrophobic portion may extend into the non-aqueous phase. Thus, the amphiphilic species can spontaneously organize under certain conditions so that the amphiphilic species molecules orient substantially parallel to each other and are oriented substantially perpendicular to the interface between two adjoining fluids, such as an inner droplet and outer droplet, or an outer droplet and an outer fluid. As the amphiphilic species become organized, they may form a sheet, e.g., a substantially spherical sheet, with a hydrophobic surface and an opposed hydrophilic surface. Depending on the arrangement of fluids, the hydrophobic side may face inwardly or outwardly and the hydrophilic side may face inwardly or outwardly. The resulting multiple emulsion structure may be a bilayer or a multi-lamellar structure.

Various matrix-forming polymers can be used for the polymersomes, thus allowing control of properties such as the biodegradability, thermoresponsiveness, photoresponsiveness, elasticity, and surface chemistry.

The polymers used to form the polymersome shell from the middle fluid of the emulsion can be biocompatible and/or biodegradable. “Biocompatible” refers to a polymer that does not have toxic or injurious effects on biological function and/or living cells and/or tissue. “Biodegradable” refers to polymers that are capable of being broken down into innocuous products by the action of living cells, such as microorganisms. Exemplary biocompatible and/or biodegradble polymers include polylactic acid (PLA), Poly(ε-caprolactone) (PCL), Polylactic acid co-glycolic acid (PLGA), Polyhydroxy butyrate (PHB), poly(ortho esters) (POE) and Poly-Hydroxybutyrate-co-b-Hydroxy valerate (PHBV). Other polymers used for making polymersomes include poly(ethylene glycol) (PEG/PEO), poly(2-methyloxazoline), polydimethulsiloxane (PDMS), and poly(methyl methacrylate) (PMMA).

The thermoresponsiveness of polymersomes can be controlled using various types and amounts of one or more polymers. In an embodiment, the polymers used are one or more diblock copolymers such as poly(ethylene glycol)-b-poly(lactic acid) (PEG-b-PLA) or poly(N-isopropylacrylamide)-bpoly(lactic-co-glycolic acid) (PNIPAM-b-PLGA). In an embodiment, the percentage of one diblock copolymer is about 1, 2, 5, 6, 7, 8, 9, 10, 15, or 20 wt % of the total matrix-forming polymer.

The photoresponsiveness of the polymersomes can be tuned by adding, for example, dodecylthiol-stabilized gold nanoparticles. Additionally, the elasticity of the polymersomes can be controlled, for example, by synthesizing biodegradable latent acid polymers with diol co-precursors. Thus, polymersome shells can range from hard, solid materials to viscous fluid-like materials. In one embodiment, the elasticity of the polymersome is similar to that of red blood cells, which is less than or equal to about 50 kPa.

The degradation rates of the polymersomes can be controlled using different ratios of biodegradable block co-polymers. In one embodiment, the degradation rate is less than about 1 hour, 6 hours, 12 hours, 1 day, 5 days, 10 days, 15 days, 20 days, 30 days, 2 months, 3 months, 4 months, 5 months, 6 months, 7 months, 8 months, 9 months, 10 months, 11 months, or 12 months. For example, 90:10 poly([rac-lactide]-co[ε-caprolactone]) degrades in about 2 months. By increasing the ratio of one polymeric block, such as PCL, the degradation time increases to about one year. In another embodiment, the degradation rates can be tuned by synthesizing biodegradable latent acid polymers using different ratios of diol and ether lactide precursors; this synthesis approach provides precise control of alpha hydroxyl acid segments in the polymer that controls the erosion rate.

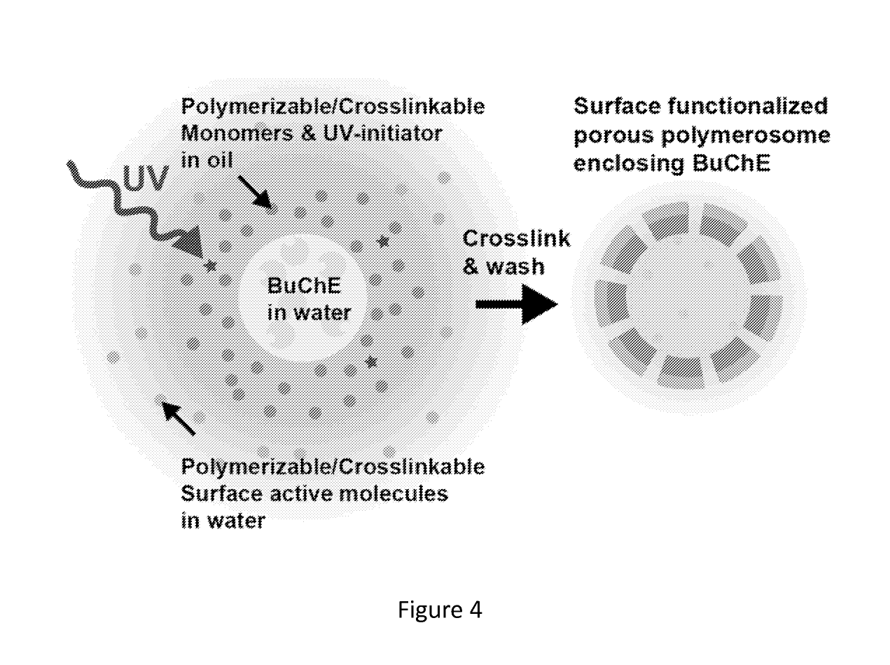

The surface chemistry of the polymersomes can also be adjusted. To facilitate long circulation times in the blood stream and inhibit phagocytosis of the polymersomes, the polymers can be modified with different functional moieties such as carboxyl or amine groups and attach PEG and inhibitory bio molecules such as CD47 to the capsule surface using various coupling reactions. Amine groups can be introduced in the particles by coupling using amine-reactive compounds, such as NHS ester methyl-capped PEG. Alternatively, PEG functionalized with acrylic groups can be dispersed in the aqueous continuous fluid and linked to the surface of the polymer containing only acrylic groups during in-situ photopolymerization (FIG. 4).

In some embodiments, a specific shell material may be chosen to dissolve, rupture, or otherwise release its contents under certain conditions. For example, if a polymerosome contains a drug, the shell components may be chosen to dissolve under certain physiological conditions (e.g., pH, temperature, osmotic strength), allowing the drug to be selectively released.

Pores can be formed within the polymersome shell using photocurable polymers or with the use of pore forming agents (porogen). In one embodiment, the polymers are functionalized for linkage and pore formation via in-situ photopolymerization. For example, acrylate and methacrylate groups can be added using methacryloyl chloride to covalently link the groups to the polymer. Photoinitiators can be used in the middle fluid or in both the middle and outer fluids. Suitable photoinitiators include, for example, 2,2-Dimethoxy-2-phenylacetophenone, Bis(2,4,6-trimethylbenzoyl)-phenylphosphineoxide, 4-(2-hydroxyethoxyl)phenyl-(2-hydroxy-2-propyl)ketone. The existence of covalent crosslinking bounds within the polymer backbone can be confirmed using, for example, Fourier transform infrared spectroscopy (FTIR). In another embodiment, a porogen templating strategy is used to form pores. Here, the functionalized polymers are dispersed in a non-reactive solvent, which serves as the porogen solvent. Upon UV exposure, precipitation polymerization occurs to form phase separated domains of crosslinked polymer and liquid porogen. Such a porogen solvent should be non-halogenated as to not hinder radical polymerization and should have a low boiling point to facilitate selective removal after membrane consolidation. Exemplary solvents include hexane, cyclohexane, 1,4-dioxane, ethers, and tetrahydrofuran. By controlling the ratio of dispersed polymer to porogen solution the shell thickness as well as membrane pore size can be controlled. In yet another embodiment, low molecular weight liquid acrylic monomers or oligomers can be used, which allows for applying a much wider range of monomer to porogen ratio than is possible using large molecular weight precursors. In an embodiment, the porogen is a non-halogenated hydrocarbon oils with high boiling points. Pore size distribution can be characterized using gaseous physisorption analysis of polymersomes which have been freeze-dried.

Polymersome diameter sizes can range from about 1-200 μm, 1-100 μm, 1-80 μm, 1-50 μm, 1-30 μm, 20-40 μm, 1-10 μm, or 1-5 μm, or a mean diameter of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100 μm. Ranges and values intermediate to the above recited ranges and values are also contemplated to be part of the invention.

In one embodiment, dewetting to remove of a portion of the middle fluid after the formation of a multiple emulsion can accomplished by removing from the fluid, in part or in whole, a component of the middle fluid, such as a solvent or carrier, through evaporation or diffusion. The remaining component or components of the middle fluid may self-organize or otherwise harden as a result of the reduction in the amount of solvent or carrier in the middle fluid, similar to those processes previously described, resulting in a polymersome. This shell formation can occur, for example, through crystallization or self-assembly of polymers dissolved in the middle fluid. For instance, a surfactant or surfactants can be used so that when the surfactant concentration in the middle fluid increases (e.g., concurrently with a decrease in the solvent concentration) the surfactant molecules are oriented so that like regions of the surfactant are associated with the inner droplet and/or the outer fluid. Within the shell itself (i.e., the middle fluid), different regions of the surfactant molecules may associate with each other, resulting in a concentrating of materials that then form a membrane of lamellar sheet(s) composed primarily or substantially of surfactant. The membrane may be solid or semi-solid in some cases. Non-surfactants can also be used.

In cases where it may be desirable to remove a portion of the middle fluid from the outer drop, for example, when forming a shell through self-assembly, some of the components of the middle fluid may be at least partially miscible in the outer fluid. This can allow the components to diffuse over time into the outer solvent, reducing the concentration of the components in the outer droplet, which can effectively increase the concentration of any of the immiscible components, e.g., polymers or surfactants, that comprise the outer droplet. This can lead to the self-assembly or gelation of polymers or other shell precursors in some embodiments, and can result in the formation of a solid or semi-solid shell. During droplet formation, it may still be preferred that the middle fluid be at least substantially immiscible with the outer fluid. This immiscibility can be provided, for example, by polymers, surfactants, solvents, or other components that form a portion of the middle fluid, but are not able to readily diffuse, at least entirely, into the outer fluid after droplet formation. Thus, the middle fluid can include, in certain embodiments, both a miscible component that can diffuse into the outer fluid after droplet formation, and an immiscible component that helps to promote droplet formation.

b. Liposomes

When other species such as lipids or phospholipids are used as the middle fluid in a emulsion, the resulting droplets can be referred to as liposomes (lipid vesicles). As used herein, the term “liposome” refers to a vesicle composed of amphiphilic lipids arranged in at least one bilayer, e.g., one bilayer or a plurality of bilayers. Liposomes include unilamellar and multilamellar vesicles that have a membrane formed from a lipophilic material and an aqueous interior. The aqueous portion contains the active agent. The lipophilic material isolates the aqueous interior from an aqueous exterior, which typically does not include the active agent composition. Liposomes are useful for the transfer and delivery of active ingredients to the site of action. The lipophilic material can be composed of one or more types of lipids, which can be either synthetic, naturally occurring, or a combination of both.

In one embodiment, an asymmetric liposome is provided, i.e., a liposome comprising a lipid bilayer having a first, inner surface comprising a first lipid composition and a second outer surface comprising a second lipid composition distinguishable from the first lipid composition, where the first, inner surface and the second, outer surface together form a lipid bilayer membrane defining the liposome, or at least one shell of the liposome if the liposome is a multilamellar liposome. Such a liposome may be formed, for example, by incorporating a first lipid in a first droplet and a second lipid in a second droplet surrounding the first droplet in a multiple emulsion, then removing the solvent from the shell using techniques such as evaporation or diffusion, leaving the lipids behind. As mentioned, higher degrees of nesting, i.e., to produce multilamellar liposomes, can also be fabricated, e.g., a first shell of a liposome may comprise a first, inner surface comprising a first lipid composition and a second outer surface comprising a second lipid composition distinguishable from the first lipid composition, and a second shell comprising a first, inner surface comprising a third lipid composition and a second outer surface comprising a fourth lipid composition distinguishable from the third lipid composition.

A liposome containing an active agent can be prepared by a variety of methods. For example, lipids can be dissolved in, for example, a chloroform/methanol solution (e.g. 1:2, v/v) and rotary evaporated to dryness under reduced pressure to form a dry lipid film. Addition of the active agent solution is then added to the dry lipid film and vigorously agitated for a few minutes and subjected to further incubation in a shaker bath. Centrifugation can be used to separate the liposomes from excess unencapsulated enzyme and resuspending the pellet to a desired final volume.

In another example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The active agent preparation is then added to the micelles that include the lipid component. The groups on the lipid interact with the active agent and condense around the active agent to form a liposome. After condensation, the detergent is removed, e.g., by dialysis, to yield a liposomal preparation of an active agent.

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Further advantages of liposomes include: liposomes obtained from natural phospholipids are biocompatible and biodegradable; liposomes can incorporate a wide range of water and lipid soluble drugs; liposomes can protect encapsulated active agents in their internal compartments from metabolism and degradation. Important considerations in the preparation of liposome formulations are the lipid surface charge, vesicle size and the aqueous volume of the liposomes.

Liposomes that include the active agent can be made highly deformable. Such deformability can enable the liposomes to penetrate through pore that are smaller than the average radius of the liposome. For example, transfersomes are a type of deformable liposomes that they are easily able to penetrate through pores which are smaller than the droplet. Transferosomes can be made by adding surface edge activators, usually surfactants, to a standard liposomal composition. Transfersomes that include an active agent can be delivered, for example, subcutaneously by infection in order to deliver the active agent to keratinocytes in the skin. In order to cross intact mammalian skin, lipid vesicles must pass through a series of fine pores, each with a diameter less than 50 nm, under the influence of a suitable transdermal gradient. In addition, due to the lipid properties, these transferosomes can be self-optimizing (adaptive to the shape of pores, e.g., in the skin), self-repairing, and can frequently reach their targets without fragmenting, and often self-loading.

c. Colloidosomes

In another embodiment, the emulsions can produce a colloidosome, i.e., a fluidic droplet surrounded by a shell of colloidal particles, which have been coagulated or fused. Such a colloidosome can be produced, for example, by providing colloidal particles in a shell of a multiple emulsion droplet (e.g., in an outer droplet), then removing the solvent can be removed from the shell using techniques such as evaporation or diffusion, leaving the colloids behind to form the colloidosome. Nested colloidosomes can also be produced in some cases, i.e., a colloidosome having at least a first particle shell and a second particle shell surrounding the first particle shell. The shells may or may not have the same composition of colloids. Such a nested colloidosome can be produced, according to one set of embodiments, by producing a multiple emulsion having an inner droplet, a middle droplet, and an outer droplet (etc., if higher degrees of nesting are desired), where some or all of the middle droplet(s) and outer droplets contain colloidal particles. Next, the solvents can be removed from the shells using techniques such as evaporation or diffusion, leaving behind multiple layers of colloids to from the nested colloidosome. Methods of producing colloidosomes can be found, for example, in Patent Application US20100213628, incorporated herein by reference.

d. Nanocapsules and Microcapsules

The porous particles can also be in the form of microcapsules or nanocapsules.

The term “nanocapsule” refers to particles having a size (e.g., a diameter) between 1 nm and 1,000 nm; or between 1 nm and 600 nm; or between 50 nm and 500 nm; or between 100 nm and 400 nm; or between 150 nm and 350 nm; or between 200 nm and 300 nm. In certain embodiments, a “nanocapsule composition” as used herein refers to a composition that includes particles wherein at least 30%; or at least 40%; or at least 50%; or at least 60%; or at least 65%; or at least 70%; or at least 75%; or at least 80%; or at least 85%; or at least 87%; or at least 90%; or at least 92%; or at least 95%; or at least 97% of the particles fall within a specified size range, for example wherein the size range is between 1 and 1,000 nm; or between 1 nm and 600 nm; or between 50 nm and 500 nm; or between 100 nm and 400 nm; or between 150 nm and 350 nm; or between 200 nm and 300 nm.

The term “microcapsule” refers to particles having a size (e.g., a diameter) between 1 μm and 1,000 μm; or between 1 μm and 500 μm; or between 1 μm and 100 μm; or between 1 μm and 50 μm; or between 2 μm and 30 μm; or between 3 μm and 30 μm; or between 3 μm and 10 μm. In certain embodiments, a “microcapsule composition” as used herein refers to a composition that includes particles wherein at least 30%; or at least 40%; or at least 50%; or at least 60%; or at least 65%; or at least 70%; or at least 75%; or at least 80%; or at least 85%; or at least 87%; or at least 90%; or at least 92%; or at least 95%; or at least 97% of the particles fall within a specified size range, for example wherein the size range is between 1 μm and 1,000 μm; or between 1 μm and 500 μm; or between 1 μm and 100 μm; or between 1 μm and 50 μm; or between 2 μm and 30 or between 3 μm and 30 μm; or between 3 μm and 10 μm.

Microcapsules and/or nanocapsules as described herein may be made or manufactured using any technique known in the art, including emulsification techniques (including double-emulsification techniques), spray drying techniques, water-in-oil-in-water techniques, syringe extrusion techniques, coaxial air flow methods, mechanical disturbance methods, electrostatic force methods, electrostatic bead generator methods, and/or droplet generator methods. For example, microcapsules and/or nanocapsules may be manufactured using techniques and methods similar to those described in U.S. Pat. No. 6,884,432, hereby incorporated by reference in its entirety. Components of microcapsules and nanocapsules are described, for example, in U.S. Patent Publication No. US20120219629 and US20110195030, hereby incorporated by reference in their entirety. In certain embodiments, microcapsules or nanocapsules may be gelatin-based; for example similar to those disclosed in Vandelli, et al., International Journal of Pharmaceutics (2001), 215:175-185. In various embodiments, microparticles and or nanoparticles include a gel or matrix having the monomers, polymers and/or polymerization initiators as described in US20120219629. The size and other properties of microcapsules and nanocapsules may be changed by altering various parameters in the production process. Freidberg et al., (2004) 282:1-18 (hereby incorporated by reference in its entirety) provides a review of procedures and compositions for microsphere manufacture, any of which procedures and compositions may be used in conjunction with microcapsules or nanocapsules of the present technology.

e. Micro- and Nano-Gels

The terms “microgel” and “nanogel” mean a water soluble polymer cross-linked to form a microparticle or nanoparticle, either in solid or capsule form. The micro- or nanogels may form a colloidal network when placed in a suitable medium, such as water. Micro- and nanogels are further described in US20110287262, hereby incorporated by reference in their entirety.

2. Micelles

In one embodiment, a porous particle suitable comprising an active agent for use in the nanostructured active therapeutic vehicles of the present invention is a micelle. “Micelles” are a particular type of molecular assembly in which amphiphilic molecules are self-assembled and arranged in a spherical structure. In aqueous environments, the hydrophobic portions of the molecules are directed inward forming the micelle core, used to hold active agents which may be poorly soluble or protect the active agent from destruction in biological surroundings, and leaving the hydrophilic portions in contact with the surrounding aqueous phase. The converse arrangement exists if the surrounding environment is hydrophobic. Micelles generally range between 5 to 100 nm. Micelles can be prepared from polymers, lipids, or polymer-lipid combinations. Depending on the molecules used to prepare the micelles, the stability of the micelles can be tuned.

In one embodiment, polymer micelles are used and prepared from self-assembly of amphiphilic block or graft co-polymers in aqueous media, producing nanoparticles with hydrophobic cores for encapsulation of the active agent and hydrophilic shells for stabilization and specific targeting.

The hydrophilic shell can be selectively cross-linked to improve the structure integrity of polymer micelles. The micelles can also be made suitable for biomedical applications by tuning its properties such that the micelles are thermoresponsive, pH-responsive, and/or biodegradable.

The surface of the micelles can be modified to alter a nanoparticle's effective exterior. For example, PEGylation can be used, for example, to solubilize the micelle carrier, to protect the active agent from enzymes, to prevent an immune response, and/or to hinder renal excretion. Targeting ligands can similarly be added to increase the active agent's effective concentration at a desired site. Thus, targeting can be achieved both passively (via enhanced permeation and retention) and actively (via the conjugation of molecular homing devices).

Micelles can be prepared by known methods from amphiphilic components (such as lipidated polymer) combined with various poorly soluble pharmaceutical agent in a form of mechanical mixture (e.g., warming, shaking, stirring or ultrasound treatment) that spontaneously self-assembles in aqueous media. Alternatively, any known method of mixing solid ingredients may be applied. These methods include, for example, direct dissolution or dialysis of an amphiphile solution in a water-miscible organic solvent against aqueous medium. The organic solvent may be removed by evaporation. An excess of a poorly soluble agent that does not incorporate into micelles, may be removed by filtration and/or centrifugation. Resultant particles consist of a hydrophobic core made of water-insoluble fragments of amphiphilic molecules and poorly soluble drug surrounded by a protective shell formed by the water-soluble parts of amphiphilic molecules.

Conjugates of lipid residues with water-soluble polymers are another example of the micelle of the invention. In this case, the lipid and polymer parts are covalently attached to each other forming lipid-polymer block co-polymer. Examples of suitable lipids include, but are not limited to, saturated or non-saturated 18-28 carbon atoms long hydrocarbon chains fatty acids and phospholipids with saturated and non-saturated acyl chains with the length from 12 to 22 carbon atoms, linear or branched. In one embodiment, the lipid is a diacyllipid, e.g., phosphatidylethanolamine. Examples of water-soluble polymers include, but are not limited to, PEG with molecular weights in the range between 500 to 10,000 daltons or between 1,000 to 8,000 daltons, with straight or branched polymer chains. In addition to amphiphilic components, lipids not carrying polymer part may also be included into particle composition yielding mixed micelles.

Micelles can be prepared from lipids or polymers. Exemplary polymers include poly(D,Llactide)-graft-poly(N-isopropyl acrylamide-co-methacrylic acid) (PLA-g-P(NIPAm-co-MAA)) to yield a hydrophilic outer shell and a hydrophobic inner core that exhibited a phase transition temperature above 37° C. For example, micelles can be prepared from conjugates of polyethyleneglycol (PEG) and diacyllipids, such as phosphatidylethanolamine (PE).

Micelle forming compounds may be added and include, for example, lecithin, hyaluronic acid, pharmaceutically acceptable salts of hyaluronic acid, glycolic acid, lactic acid, chamomile extract, cucumber extract, oleic acid, linoleic acid, linolenic acid, monoolein, monooleates, monolaurates, borage oil, evening of primrose oil, menthol, trihydroxy oxo cholanyl glycine and pharmaceutically acceptable salts thereof, glycerin, polyglycerin, lysine, polylysine, triolein, polyoxyethylene ethers and analogues thereof, polidocanol alkyl ethers and analogues thereof, chenodeoxycholate, deoxycholate, and mixtures thereof. Phenol and/or m-cresol may be added to the mixed micellar composition to stabilize the formulation and protect against bacterial growth. Alternatively, phenol and/or m-cresol may be added with the micelle forming ingredients. An isotonic agent such as glycerin may also be added after formation of the mixed micellar composition.

Exemplary cationic lipids include N,N-dioleyl-N,N-dimethylammonium chloride (DODAC), N,N-distearyl-N,N-dimethylammonium bromide (DDAB), N-(I-(2,3-dioleoyloxy)propyl)-N,N,N-trimethylammonium chloride (DOTAP), N-(I-(2,3-dioleyloxyl)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyl)tetrahydro-3aH-cyclopenta[d][1,3]dioxol-5-amine (ALN100), (6Z,9Z,28Z,31Z)-heptatriaconta-6,9,28,31-tetraen-19-yl 4-(dimethylamino)butanoate (MC3), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (Tech G1), or a mixture thereof.

The ionizable/non-cationic lipid can be an anionic lipid or a neutral lipid including, but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof.

The conjugated lipid that inhibits aggregation of particles can be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate can be, for example, a PEG-dilauryloxypropyl (Ci2), a PEG-dimyristyloxypropyl (Ci4), a PEG-dipalmityloxypropyl (Ci6), or a PEG-distearyloxypropyl (C]8). The conjugated lipid that prevents aggregation of particles can be from 0 mol % to about 20 mol % or about 2 mol % of the total lipid present in the particle.

3. Dendrimers

In one embodiment, a porous particle suitable comprising an active agent for use in the nanostructured active therapeutic vehicles of the present invention is a dendrimer. Dendrimers are a family of nanosized, three-dimensional polymers characterized by a unique tree-like branching architecture and compact spherical geometry in solution, and are obtained by a reiterative sequence of reactions. Dendrimers are composed of individual “wedges” or dendrons that radiate from a central core where each layer of concentric branching units constitutes one complete generation (G) in the dendrimer series and is identified with a specific generation number. This branching architecture leads to a controlled incremental increase in a dendrimer's molecular weight, size, and number of surface groups. The dendrimer family includes poly(amidoamine) (PAMAM) dendrimers, biodegradable dendrimers, amino acid-based dendrimers, glycodendrimers, hydrophobic dendrimers, and asymmetric dendrimers.

Each monomer unit is added to a branching point to yield a spherical polymer with a large number of surface groups. Each successive layer of branching units constitutes a new generation (G) with a specific number in the dendrimer series. Dendrimers are routinely synthesized as tunable particles that may be designed and regulated as a function of their size, shape, surface chemistry and interior void space. Dendrimers can be obtained with structural control approaching that of traditional biomacromolecules, such as DNNPNA or proteins and are distinguished by their precise nanoscale scaffolding and nanocontainer properties. Dendrimers are microscopic particles with at least one nanoscale dimension, usually less than 100 nm. Dendrimers may have a size of about 1 nm-0.4 um.

Synthesis of PAMAM dendrimers is initiated using an alkyldiamine core (e.g., ethylene diamine; EDA), which reacts via Michael addition with methyl acrylate monomers to produce a branched intermediate that can be transformed to the smallest generation of PAMAM dendrimers with NH2, OH, or COOH surface groups. The reaction of this branched intermediate with excess EDA produces G0 with four NH₂ surface groups. Similarly, the reaction of the same intermediate with ethanolamine produces G0 with four OH surface groups. Hydrolysis of the methyl ester in this intermediate produces the smallest anionic dendrimer (G0.5) with four COOH groups. Synthesis of higher generations of PAMAM dendrimers is achieved by sequential Michael addition of methyl acrylate monomers followed by an exhaustive amidation reaction with EDA. This synthesis method produces highly organized and relatively monodisperse polymers that display a controlled incremental increase in size, molecular weight, and number of surface groups with the increase in generation number.

Biodegradable dendrimers are commonly prepared by inclusion of ester groups in the polymer backbone, which will be chemically hydrolyzed and/or enzymatically cleaved by esterases in physiological solutions. An example of a biodegradable dendrimers is a polyester dendrimers [poly(glycerol-succinic acid); PGLSA].

Glycodendrimers can be prepared by functionalizing the surface groups of G2-G4 PAMAM dendrimers with sugars such as lactose and maltose sugars, R-amino acid derivatives, N-carboxyanhydride (glycoNCA) glucose and N-acetyl-D-glucosamine ligands. Other glycodendrimers have been synthesized by coupling isothiocyanate functionalized glycosyl and mannopyranoside ligands as well as an N-hydroxysuccinimide (NHS) activated galactopyranosyl derivative to amine-terminated dendrimers.

Symmetry of dendrimer's architecture is a result of the controlled iterative synthetic steps, which produces highly monodisperse and symmetrical polymers. However, imparting asymmetry to dendrimer's architecture can provide a range of novel structures, which may favorably affect their pharmacokinetic profile in vivo. Asymmetric dendrimers are synthesized by coupling dendrons of different generations to a linear core, which yields a branched dendrimer with a nonuniform orthogonal architecture. This asymmetry allows for tunable structures and molecular weights, with precise control over the number of functional groups available on each dendron for attachment of drugs, imaging agents, and other therapeutic moieties.

4. Other Particles

Other particles such as carbon and silica can be made into porous materials or to possess porous structures. For example mesostructured silica spheres with large pores using micelles as the template have been prepared (see, e.g., Lefèvre B., et al. Chem. Mater., 2005, 17, 601). Template carbonization methods allow carbon structure to be controlled in terms of various aspects such as pore structure, graphitizability and microscopic morphology. Some methods require template removal treatment. Other methods such as the polymer blend carbonization method does not require such treatment, because the pyrolyzing polymer will decompose spontaneously during carbonization. Organic compounds as a template has been performed for the production of mesoporous silica such as MCM-41 and FMS-16, which contain hexagonally arranged one-dimensional pores of tunable diameter from 1.5 to 10 nm. These mesoporous silica were prepared through a liquid crystal templating mechanism where organic surfactant molecules are self-assembled into a hexagonal arrangement of rod-like micelles and these organic rods function as a template during the formation of the silica network structure. Final heat-treatment of the silica complex at a high temperature converts the rod-like micelles into the one-dimensional pores. Such structurally regulated micelles of organic surfactants might be utilized as a template in a new type of template carbonization method. Control or pore structure in carbon materials have been described in, for example, Kyotani, 2000, Carbon, 38: 269-286.

### B. Methods for Fabricating Biodegradable Polymeric Fibers and Threads

The nanostructured active therapeutic vehicles of the present invention comprise a biodegradable polymer fiber and/or thread. The terms “fiber” and “polymeric fiber” are used herein interchangeably, and both terms refer to fibers having micron, submicron, and nanometer dimensions. A “polymeric thread” or “thread”, as used herein, is a tightly twisted strand of two or more polymeric fibers.

Devices and methods of use thereof for the fabrication of biodegradable polymeric fibers and threads suitable for use in the present invention are described in, for example, U.S. Patent Publication Nos. U.S. 2012/0135448 and U.S. 2013/0312638, the entire contents of each of which are incorporated herein by reference. These devices, referred to as Rotary Jet Spinning Devices (RJS) and use of such devices, allow the facile fabrication of polymeric fibers and threads having micron, submicron, and nanometer dimensions with tunable orientation, alignment, and diameter by applying centrifugal or rotational motion to a polymer and without use of an electrical field, e.g., a high voltage electrical field, and/or needle. RJS devices and use of such devices methods permit the formation of polymeric fibers and threads by essentially ejecting a polymer solution through an orifice of a reservoir into air. Air drag extends and elongates the jets into fibers and threads as the solvent in the material solution rapidly evaporates.

Briefly, RJS systems and devices include a reservoir for holding a polymer, the reservoir including one or more orifices for ejecting the polymer during fiber and/or thread formation, thereby forming a micron, submicron or nanometer dimension polymeric fiber and/or thread and a collection device for accepting the formed micron, submicron or nanometer dimension polymeric fiber and/or thread, wherein at least one of the reservoir and the collection device employs rotational motion during fiber and/or thread formation. The device may include a rotary motion generator for imparting a rotational motion to the reservoir and/or to the collection device.

The devices may further comprise a component suitable for continuously feeding the polymer into the rotating reservoir, such as a spout or syringe pump

The RJS device (and/or the collection device) may be maintained at about room temperature, e.g., about 20, 21, 22, 23, 24, 25, 26, 27, 28, 29, or about 30° C. and ambient humidity, e.g., about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or about 90% humidity. The devices may be maintained at and the methods may be formed at any suitable temperature and humidity depending on the desired surface topography of the polymeric fibers and/or thread to be fabricated. For example, increasing humidity from about 30% to about 50% results in the fabrication of porous fibers and/or threads, while decreasing humidity to about 25% results in the fabrication of smooth fibers and/or threads. As smooth fibers and/or threads have more tensile strength than porous fibers and/or threads, in one embodiment, the devices of the invention are maintained and the methods performed in controlled humidity conditions, e.g., humidity varying by about less than about 10%.

The reservoir may also include a heating element for heating and/or melting the polymer.

The reservoir may have a volume ranging from about one nanoliter to about 1 milliliter, about one nanoliter to about 5 milliliters, about 1 nanoliter to about 100 milliliters, or about one microliter to about 100 milliliters, for holding the polymer. Exemplary volumes intermediate to the recited volumes are also part of the invention. In certain embodiments, the volume of the reservoir is less than about 5, less than about 4, less than about 3, less than about 2, or less than about 1 milliliter. In other embodiments, the physical size of an unfolded polymer and the desired number of polymers that will form a fiber and/or thread dictate the smallest volume of the reservoir.

Rotational speeds of the reservoir and/or collection device may range from about 3,000 rpm to about 400,000 rpm, e.g., about 3,000, 5,000, 10,000, 50,000, 55,000, 60,000, 65,000, 70,000, 75,000, 80,000, 85,000, 90,000, 95,000, 100,000, 105,000, 110,000, 115,000, 120,000, 125,000, 130,000, 135,000, 140,000, 145,000, 150,000 rpm, about 200,000 rpm, 250,000 rpm, 300,000 rpm, 350,000 rpm, or 400,000 rpm. Ranges and values intermediate to the above recited ranges and values are also contemplated to be part of the invention.

Rotational motion may be provided for a time sufficient to form a desired polymeric fiber and/or thread, such as, for example, about 1 minute to about 100 minutes, about 1 minute to about 60 minutes, about 10 minutes to about 60 minutes, about 30 minutes to about 60 minutes, about 1 minute to about 30 minutes, about 20 minutes to about 50 minutes, about 5 minutes to about 20 minutes, about 5 minutes to about 30 minutes, or about 15 minutes to about 30 minutes, about 5-100 minutes, about 10-100 minutes, about 20-100 minutes, about 30-100 minutes, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 minutes, or more. Times and ranges intermediate to the above-recited values are also intended to be part of this invention.

One or more jets of a polymer solution may be ejected from one or more reservoirs containing the material solution, and one or more air foils may be used to modify the air flow and/or air turbulence in the surrounding air through which the jets of the polymer solution descend which, in turn, affects the alignment of the fibers and/or threads that are formed from the jets.

An “air foil” refers to a single-part or multi-part mechanical member disposed or formed in the vicinity of one or more reservoirs to modify the air flow and/or the air turbulence in the surrounding air experienced by a material solution ejected from the reservoirs.

An exemplary air foil may be provided vertically above, vertically below, or both vertically above and below one or more orifices of a reservoir. Depending on the geometry and position of an exemplary air foil relative to the reservoir, the air flow created by the air foil may push fibers formed and/or threads by an RJS device upward or downward along the vertical direction. An air foil may be stationary or moving.

In some embodiments, the reservoir may not be rotated, but may be pressurized to eject the polymer solution from the reservoir through one or more orifices. For example, a mechanical pressurizer may be applied to one or more surfaces of the reservoir to decrease the volume of the reservoir, and thereby eject the polymer solution from the reservoir. In other embodiments, a fluid pressure may be introduced into the reservoir to pressurize the internal volume of the reservoir, and thereby eject the polymer solution from the reservoir.

The orifices may be provided on any surface or wall of the reservoir, e.g., side walls, top walls, bottom walls, etc. When multiple orifices are provided, the orifices may be grouped together in close proximity to one another, e.g., on the same surface of the reservoir, or may be spaced apart from one another, e.g., on different surfaces of the reservoir. The orifices may be of the same diameter or of different diameters, the same length or of different lengths.

Exemplary orifice lengths that may be used range from between about 0.001 m and about 0.1 m, e.g., 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, or 0.1 m. Ranges and values intermediate to the above recited ranges and values are also contemplated to be part of the invention.

Exemplary orifice diameters that may be used range between about 0.1 μm and about 1000 μm, e.g., 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or about 1000 μm. Ranges and values intermediate to the above recited ranges and values are also contemplated to be part of the invention.

One or more nozzles may be provided associated with one or more orifices of a reservoir through which a polymer solution is ejected from the reservoir.

The devices may also further include a control mechanism for controlling the speed of the motion imparted by the motion generator.

RJS devices may include an air vessel for circulating a vortex of air around the formed fibers to wind the fibers into one or more threads. The air vessel may include an enclosed member extending substantially vertically for accommodating the descending formed fibers, one or more angle nozzles for introduced one or more angled air jets into the enclosed member, and one or more air introduction pipes couplable to the one or more nozzles for introducing the air jets into the enclosed member. The air jets may travel vertically downward along the enclosed member substantially in helical rings.

The RJS devices may include one or more mechanical members, which may be stationary or moving, disposed or formed on or in the vicinity of the reservoir for increasing an air flow or an air turbulence experienced by the polymer ejected from the reservoir, and a collection device for accepting the formed micron, submicron or nanometer dimension polymeric fiber. The one or more mechanical members may be disposed on the reservoir.

The one or more mechanical members may be disposed vertically above the one or more orifices of the reservoir or disposed vertically below the one or more orifices of the reservoir.

The devices may further include a motion generator for imparting a motion to the reservoir, wherein the one or more mechanical members are disposed on the motion generator.

The polymeric fibers and/or threads may be of any length. In one embodiment, the length of the polymeric fibers and/or threads is dependent on the length of time the device is in motion and/or the amount of polymer fed into the system. For example, the polymeric fibers and/or threads may be about 1 nanometer, about 10 feet, or about 500 yards. Additionally, the polymeric fibers and/or threads may be cut to a desired length using any suitable instrument.

Methods of forming fibers and/or threads using an RJS device include feeding a polymer into a reservoir of an RJS device and providing motion at a speed and for a time sufficient to form a micron, submicron or nanometer dimension polymeric fiber and/or threads. Methods for forming polymeric fibers and/or threads may also include providing a volume of a polymer solution (e.g., a natural polymer) and imparting a shear force (e.g., sufficient to expose molecule-molecule, e.g., protein-protein, binding sites in the polymer, thereby facilitating unfolding of the polymer and inducing fibrillogenesis) to a surface of the polymer solution such that the polymer in the solution is unfolded, thereby forming a fiber and/or thread.

When the polymer comprises a natural polymer, such as a protein, because the polymeric fibers come into contact with each other in an extended state during fiber fabrication in a RJS device, the natural polymeric fibers relax after winding and by controlling the solvent evaporation rate of the polymer solution (using, e.g., an air foil or jet, controlling polymer solution concentrations, speed and/or time of rotation), a covalently bound thread whose strength to diameter or cross-sectional area ratio far exceeds conventional threads or fibers is created.

Alternatively, threads of polymeric fibers may be fabricated by spinning fibers together using conventional thread making processes.

A polymer for use in the methods of the invention may be fed into the reservoir as a polymer solution. Accordingly, methods for fabricating a polymeric fiber and/or thread may include dissolving the polymer in an appropriate solvent (e.g., chloroform, water, ethanol, isopropanol) prior to feeding the polymer into the reservoir.

Alternatively, the polymer may be fed into the reservoir as a polymer melt and, thus, the reservoir may be heated at a temperature suitable for melting the polymer, e.g., heated at a temperature of about 100° C.-300° C., 100° C.-200° C., about 150-300° C., about 150-250° C., or about 150-200° C., 200° C.-250° C., 225° C.-275° C., 220° C.-250° C., or about 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or about 300° C. Ranges and temperatures intermediate to the recited temperature ranges are also part of the invention. In such embodiments, the reservoir may further comprise a heating element.

The polymeric fibers and/or threads may be contacted with an agent to produce or increase the size of pores or number of pores per surface unit area in the polymeric fibers and/or threads.

In certain embodiments of the invention, in addition to mixing a porous particle comprising an active agent with the fibers and/or threads, the methods may include mixing one or more additional biologically active agents, e.g., a polypeptide, protein, nucleic acid molecule, nucleotide, lipid, biocide, antimicrobial, or pharmaceutically active agent, with the polymer during the fabrication process of the polymeric fibers.

The fibers and/or threads (as well as the nanostructured active therapeutic vehicles) may be collected from the collection device using any suitable technique. One collection technique involves manually extracting the fibers from the collection device. Another collection technique involves the use of a spinning mandrill to wind the fibers and/or threads to remove them from the collection device. Yet another collection technique involves emptying the collection device, manually or mechanically. In some embodiments, the collected fibers and/or threads may be mechanically manipulated to adjust the alignment of the fibers and/or threads and to achieve a desired orientation of the fibers, e.g., by applying uniaxial tension, biaxial tension, and/or shear, and/or by spinning the fibers and/or threads onto a mandrill.

To fabricate a nanostructured active therapeutic vehicle comprising a biodegradable polymer fiber and/or thread comprising a porous particle (e.g., encapsulating an active agent), a polymeric fiber and/or thread, e.g., a plurality of polymeric fibers and/or threads, is contacted with a porous particle, e.g., a plurality of porous particles. The polymer may be contacted with a porous particle during the fabrication process such that fibers and/or threads populated with porous particles are produced, e.g., the threads and/or fibers surround, either partially or totally, the porous particles. The porous particles may be mixed with a polymer prior to, during, or after the polymer is fed into the reservoir of an RJS device, or the polymer may be contacted with the porous particles as the polymer is ejected from an orifice of a reservoir, or a polymeric fiber may be contacted with a porous particle in the collection device, or following removal from the collection device by any suitable means to, e.g., coat the polymeric fibers with the porous particles.

Any biodegradable polymer may be used to fabricate polymeric fibers and/or threads for use in the compositions and methods of the invention.

The polymers may be biocompatible and synthetic or natural polymers. Exemplary synthetic polymers include, for example, poly(urethanes), poly(siloxanes) or silicones, poly(ethylene), poly(vinyl pyrrolidone), poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactides (PLA), polyglycolides (PGA), poly(lactide-co-glycolides) (PLGA), polyanhydrides, polycaprolactones (PCL), polyphosphazenes, polygermanes, polyorthoesters, polyesters, polyamides, polyolefins, polycarbonates, polyaramides, polyimides, and copolymers and derivatives thereof.

Natural polymers e.g., biogenic polymers, include, for example, proteins, polysaccharides, lipids, nucleic acids or combinations thereof.

Exemplary natural polymers for use in the compositions and methods of the invention include, but are not limited to, e.g., fibrous proteins, extracellular matrix proteins, silk (e.g., fibroin, sericin, etc.), keratins (e.g., alpha-keratin, beta-keratin, etc.), elastins (e.g., tropoelastin, etc.), fibrillin (e.g., fibrillin-1, fibrillin-2, fibrillin-3, fibrillin-4, etc.), fibrinogen/fibrins/thrombin (e.g., fibrinogen), fibronectin, laminin, collagens (e.g., collagen I, collagen II, collagen III, collagen IV, collagen V, etc.), vimentin, neurofilaments (e.g., light chain neurofilaments NF-L, medium chain neurofilaments NF-M, heavy chain neurofilaments NF-H, etc.), amyloids (e.g., alpha-amyloid, beta-amyloid, etc.), actin, myosins (e.g., myosin I-XVII, etc.), titin, chitin, hyaluronic acid (e.g., D-glucuronic acid, D-N-acetylglucosamine, etc.), glycosaminoglycans (GAGs) e.g., heparan sulfate, chondroitin sulfate, keratin sulfate, gelatin, albumin, etc., and combinations thereof.

The polymers for use in the compositions and methods of the invention may be mixtures of two or more polymers and/or two or more copolymers. In one embodiment the polymers for use in the devices and methods of the invention may be a mixture of one or more polymers and one or more copolymers. In another embodiment, the polymers for use in the compositions and methods of the invention may be a mixture of one or more synthetic polymers and one or more naturally occurring polymers.

### C. Active Agents

As used herein the term an “active agent”, used interchangeably with the term a “therapeutically active agent” refers to any drug, pharmaceutical substance, or bioactive agent which treats and/or cures a disease or disorder, and/or inhibits the activity of a toxin.

Active agents may be low molecular weight organic compounds, e.g., small molecules, or organic macromolecules including, for example, nucleic acid based drugs (including DNA, RNA, modified DNA, modified RNA, antisense oligonucleotides, expression plasmid systems, nucleotides, modified nucleotides, nucleosides, modified nucleosides, nucleic acid ligands (e.g. aptamers), intact genes, a promotor complementary region, a repressor complementary region, an enhancer complementary region); polypeptides; peptides; proteins (including enzymes, antibodies); carbohydrates; polysaccharides and other sugars; glycoproteins, and lipids.

Examples of active agents suitable for use the present invention include an enzyme, a cytokine, a growth promoting agent, an antibody, an antigen, a hormone, a vaccine, a cell, a live-attenuated pathogen, a heat-killed pathogen, a virus, a bacteria, a fungi, a peptide, a carbohydrate, a nucleic acid, a hormone, growth factor, cytokine, interferon, receptor, antigen, allergen, antibody, antiviral, antifungal, antihelminthic, substrate, metabolite, cofactor, inhibitor, drug, nutrient, narcotic, amphetamine, barbiturate, hallucinogen, a vaccine for against a virus, bacterium, helminth and/or fungi, fragments, receptors or toxins thereof, e.g., Salmonella, Streptococcus, Brucella, Legionella, E. coli, Giardia, Cryptosporidium, Rickettsia, spore, mold, yeast, algae, amoebae, dinoflagellate, unicellular organism, pathogen, cell, combinations and mixtures thereof.

Specific examples of active agents include: steroids, respiratory agents, sympathomimetics, local anesthetics, antimicrobial agents, antiviral agents, antifungal agents, antihelminthic agents, insecticides, antihypertensive agents, antihypertensive diuretics, cardiotonics, coronary vasodilators, vasoconstrictors, β-blockers, antiarrhythmic agents, calcium antagonists, anti-convulsants, agents for dizziness, tranquilizers, antipsychotics, muscle relaxants, drugs for Parkinson's disease, respiratory agents, hormones, non-steroidal hormones, antihormones, vitamins, antitumor agents, miotics, herb medicines, herb extracts, antimuscarinics, interferons, immunokines, cytokines, muscarinic cholinergic blocking agents, mydriatics, psychic energizers, humoral agents, antispasmodics, antidepressant drugs, anti-diabetics, anorectic drugs, anti-allergenics, decongestants, expectorants, antipyretics, antimigrane, anti-malarials, anti-ulcerative, anti-estrogen, anti-hormone agents, anesthetic agent, or drugs having an action on the central nervous system.

In one embodiment, the active agent is an agent which inhibits the activity of a toxin. In one embodiment, the toxin is less than about 1 kDa, 500 Da, 300 Da, 200 Da, or about 100 Da. In another embodiment, a toxin is a cholinesterase enzyme inhibitor, such as a nerve agent or pesticide. Exemplary nerve agents include organophosphate nerve agents, for example, sarin, cyclosarin (GF), soman (GD), tabun (GA), VX, Russian-VX, novichok-5, and novichok-7. Exemplary pesticides include organophosphate pesticides, for example, paraoxan, methylparaoxan, azinphos-methyl (Gusathion, Guthion), bornyl (Swat), dimefos (Hanane, Pestox XIV), methamidophos (Supracide, ultracide), and methyl parathion (E 601, Penncap-M). In another embodiment, a toxin is cyanide or other cyanide compounds.

Active agents that inhibit the activity of a toxin include, but are not limited to, butyrylcholinesterase (BChE) which detoxifies organophosphate toxins by acting as organophosphate scavengers; phosphotriesterase enzymes, which catalyzes the detoxification of organophosphate insecticides; Hydroxocobalamin (vitamin B12a, which binds cyanide strongly to form cyanocobalamin (vitamin B12).; and Rhodanese (thiosulfate-cyanide sulfurtransferase), which is a mitochondrial enzyme that detoxifies cyanide (CN-) by converting it to thiocyanate (SCN-).

### II. Pharmaceutical Compositions

The porous particles, the biodegradable polymeric fibers and/or threads, and/or the nanostructured active therapeutic vehicles of the invention may be formulated as pharmaceutical compositions prior to contacting them with cells (in vitro or in vivo). Accordingly, in one embodiment, the present invention provides pharmaceutical compositions containing a porous particle, a biodegradable polymeric fiber and/or thread, and/or nanostructured active therapeutic vehicle, as described herein, and a pharmaceutically acceptable carrier.

The phrase “pharmaceutically acceptable” is employed herein to refer to those compounds, materials, compositions, and/or dosage forms which are, within the scope of sound medical judgment, suitable for use in contact with the tissues of human subjects and animal subjects without excessive toxicity, irritation, allergic response, or other problem or complication, commensurate with a reasonable benefit/risk ratio.

As used herein the term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions. Pharmaceutical compositions can be prepared as described above.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Supplementary active compounds can also be incorporated with the marker(s) modulator.

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; and (22) other non-toxic compatible substances employed in pharmaceutical formulations.

Pharmaceutical compositions of the invention typically must be sterile and stable under the conditions of manufacture and storage. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including an agent that delays absorption, for example, monostearate salts and gelatin.

Sterile injectable solutions can be prepared by incorporating the biodegradable polymeric fibers and/or threads, and/or nanostructured active therapeutic vehicles of the invention in the required amount in an appropriate solvent with one or a combination of ingredients enumerated above, as required, followed by sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Biodegradable polymeric fibers and/or threads, and/or nanostructured active therapeutic vehicles that can be used in the methods of the present invention include those suitable for oral, nasal, topical (including buccal and sublingual), rectal, vaginal and/or parenteral administration. The formulations may conveniently be presented in unit dosage form and may be prepared by any methods known in the art of pharmacy. The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the modulator which produces a therapeutic effect. Generally, out of one hundred percent, this amount will range from about 0.001% to about 90% of active ingredient, preferably from about 0.005% to about 70%, most preferably from about 0.01% to about 30%.

The phrases “parenteral administration” and “administered parenterally”, as used herein, means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Examples of suitable aqueous and non-aqueous carriers which may be employed along with the biodegradable polymeric fibers and/or threads, and/or nanostructured active therapeutic vehicles of the present invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Biodegradable polymeric fibers and/or threads, and/or nanostructured active therapeutic vehicles may also be administered with adjuvants such as preservatives, wetting agents, emulsifying agents and dispersing agents. Prevention of presence of microorganisms may be ensured both by sterilization procedures and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

When biodegradable polymeric fibers and/or threads, and/or nanostructured active therapeutic vehicles of the present invention are administered to humans and animals, they can be given alone or as a pharmaceutical modulator containing, for example, 0.001 to 90% (more preferably, 0.005 to 70%, such as 0.01 to 30%) of active ingredient in combination with a pharmaceutically acceptable carrier.

Biodegradable polymeric fibers and/or threads, and/or nanostructured active therapeutic vehicles can be administered with medical devices known in the art, e.g., with a needleless hypodermic injection device, such as the devices disclosed in U.S. Pat. No. 5,399,163, 5,383,851, 5,312,335, 5,064,413, 4,941,880, 4,790,824, or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medications through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known to those skilled in the art.

### III. Methods of Using the Nanostructured Active Therapeutic Vehicles

The nanostructured active therapeutic vehicles of the present invention (and pharmaceutical compositions comprising such vehicles) may be used to provide extended and sustained release of an active agent to a cell or a subject. Accordingly, the present invention provides therapeutic and prophylactic methods of use of the nanostructured active therapeutic vehicles of the invention.

For example, in one aspect, the present invention provides methods of providing sustained release of an active agent to a subject having a condition treatable with an active agent. The methods include administering to the subject an effective amount of a nanostructured active therapeutic vehicle comprising the active agent, wherein the vehicle provides sustained delivery of the active agent, e.g., for about 1 week to about 3 months, thereby providing sustained release of the active agent to the subject having a condition treatable with the active agent.

The present invention also provides methods for providing sustained release of an active agent which inhibits the activity of a toxin in a subject. The methods include administering to the subject an effective amount of a nanostructured active therapeutic vehicle comprising an active agent that inhibits the activity of the toxin, e.g. for about 1 week to about 3 months, thereby providing sustained release of an active agent which inhibits the activity of a toxin to the subject. In embodiments in which the toxin is a cholinesterase enzyme inhibitor, such as a nerve agent, and the active agent is, for example, butyrylcholinesterase (BChE), a nanostructured active therapeutic vehicle is administered to a subject subcutaneously, e.g., as a subcutaneous suture. The subcutaneously administered vehicle provides sustained release of the active agent and is useful as a prophylactic treatment for subjects at risk of being exposed to a toxin, e.g., a soldier, e.g., before a soldier goes into battle.

The activity of a toxin may also be inhibited in a cell. Accordingly, in another aspect, the present invention provides methods for inhibiting the effects of a toxin in a cell. The methods include contacting the cell with nanostructured active therapeutic vehicle comprising an active agent capable of inhibiting the activity of the toxin, thereby inhibiting the activity of a toxin in the cell.

The nanostructured active therapeutic vehicles of the present invention may contain a therapeutically effective amount or a prophylactically effective amount of the active agent.

A “therapeutically effective amount,” as used herein, is intended to include an amount of active agent effective, at dosages and for periods of time necessary, to achieve the desired result, e.g., an amount sufficient to effect treatment of the disease or disorder for which the active agent is intended to be used (e.g., by diminishing, ameliorating or maintaining the existing disease or one or more symptoms of disease). The “therapeutically effective amount” may vary depending on the active agent, how the agent is administered, the disease and its severity and the history, age, weight, family history, genetic makeup, the types of preceding or concomitant treatments, if any, and other individual characteristics of the subject to be treated. Dosage regimens may be adjusted to provide the optimum therapeutic response.

A “prophylactically effective amount,” as used herein, is intended to an amount of active agent effective, at dosages and for periods of time necessary to inhibit the activity of a toxin and/or prevent or ameliorate a disease or one or more symptoms of a disease. Ameliorating the disease includes slowing the course of the disease or reducing the severity of later-developing disease. The “prophylactically effective amount” may vary depending on the active agent, how the agent is administered, the degree of risk of disease, and the history, age, weight, family history, genetic makeup, the types of preceding or concomitant treatments, if any, and other individual characteristics of the patient to be treated.

A “therapeutically effective amount” or “prophylactically effective amount” also includes an amount of an active agent that produces some desired local or systemic effect at a reasonable benefit/risk ratio applicable to any treatment. Active agents employed in the methods of the present invention may be administered in a sufficient amount to produce a reasonable benefit/risk ratio applicable to such treatment. Dosage regimens may be adjusted to provide the optimum prohpylactic response.

As used herein, the term “subject” refers to human and non-human animals, e.g., veterinary patients. The term “non-human animal” includes all vertebrates, e.g., mammals and non-mammals, such as non-human primates, mice, rabbits, sheep, dog, cat, horse, cow, chickens, amphibians, and reptiles. In one embodiment, the subject is a human.

In certain embodiments of the invention, in which the active agent inhibits the activity of a toxin, e.g., butyrlcholinesterase, the nanostructured active therapeutic vehicles provide an activity towards the toxin, e.g., nerve agent, equivalent to that of a sustained plasma dose of about 100 mg of the active agent, e.g., butyrlcholinesterase, for an adult human.

The compositions of the invention can be administered to the subject by any route suitable for achieving the desired result(s) including, but not limited to subcutaneous, intravenous, oral, intraperitoneal, or parenteral routes, including intracranial (e.g., intraventricular, intraparenchymal and intrathecal), intramuscular, transdermal, airway (aerosol), nasal, rectal, and topical (including buccal and sublingual) administration. In certain embodiments, the compositions are administered by subcutaneous or intravenous infusion or injection. It should be noted that when a formulation that provides sustained delivery for weeks to months by the i.m or s.c./i.d. route is administered by an alternative route, there may not be sustained delivery of the agent for an equivalent length of time due to clearance of the agent by other physiological mechanisms (i.e., the dosage form may be cleared from the site of delivery such that prolonged therapeutic effects are not observed for time periods as long as those observed with i.m or s.c./i.d. injection).

In some embodiments of the invention, a nanostructured active therapeutic vehicle is administered as a pharmaceutical composition (as described above) subcutaneously to a subject. In certain embodiments of subcutaneous administration, a nanostructured active therapeutic vehicle comprises a biodegradable polymeric thread that is suitable for subcutaneous suturing.

A single dose of the nanostructured active therapeutic vehicles (and pharmaceutical compositions of the invention) provide sustained and extended release of an active agent. For example, the vehicles provide sustained release of the active agent for about 1 week, 2 weeks, 3 weeks, 4 weeks, 5 weeks, 6 weeks, 7 weeks, 8, weeks, 9, weeks, 10 weeks, 11 weeks, 12 weeks, 13 weeks, 14 weeks, 15 weeks, 16 weeks, or more.

### EXAMPLES

### Example 1

### Nanostructured Active Therapeutic Vehicles

BuChE has been shown to provide short term protection against organophophorous nerve agents in various mammals (Lenz, Maxwell et al. 2005; Lenz, Yeung et al. 2007). Yet, for BuChE to provide long term protection against nerve agents, the circulation time of the protein must be drastically increased. Extending and sustaining the circulation time should be accomplished while allowing it to bind nerve agents immediately upon exposure. Encapsulating BuChE in a conventional sealed polymerosome or liposome carrier could serve as a method for significantly extending the circulation time and furthermore facilitate oral administration of BuChE. However, such an approach requires detection of the nerve agent and release of the BuChE cargo prior to BuChE being capable of neutralizing the nerve agent. Additionally, prior to release, a threshold concentration of nerve agent is required as external triggering event. To overcome these complications, a vehicle that is purposely porous is developed. The porosity of the vehicle is optimized to concurrently allow free passage of the nerve agents while inhibiting the breakdown of BuChE by preventing the diffusion of proteins in and out of the vehicle. Selectivity can be achieved by taking advantage of the significant size difference between nerve agents (<300 Da) and proteins such as proteases (>10 kDa) (FIG. 1A). Due to the leaky nature of the polymerosomes, the administration route ensures that BuChE is not degraded prior to the vehicles entering the blood and lymph. Non-invasive oral administration, for instance, is inappropriate due to the acidic environment in the stomach. Therefore, a invasive administration methodology based on a slowly degrading suture acting as a reservoir for the polymerosomes is developed (FIG. 1B). The suture is introduced subcutaneously, and upon degradation, the particles are released and enter circulation via the lymphatic system. This methodology ensures minimal exposure of BuChE harmful environments prior to the polymerosomes entering circulation. In addition, the lifetime of the thread is tuned to ensure a protection period greatly exceeding the circulation time of individual polymerosomes. The methodology is well-suited for on-demand use in combat or disasters, especially compared to alternative invasive drug administration systems such as osmotic pumps (Gupta, Thakur et al. 2010) and microneedle therapy systems (Donnelly, Singh et al. 2010). Equally important, it offers a lower risk of infection, easy administration by the untrained user without breaking MOPP4, higher degree of control of the immune response and tissue integration, and a lower fabrication price than these established invasive delivery approaches. While the administration methodology can be used for prolonging the protection period, extending the circulation time of the individual polymerosomes by optimizing the physical and (surface)-chemical properties is an important target of the project. An increased circulation time of the individual polymerosomes will drastically limit the amount of vehicles and BuChE demanded and will furthermore enable faster degrading sutures to be employed, thereby limiting the risk of immune responses.

Based off of previously published animal studies using soluble BuChE (Lenz, Maxwell et al. 2005; Lenz, Yeung et al. 2007), the minimal concentration, C, of the circulating polymerosomes required to protect a human recipient has been estimated. In particular, it was assumed that the reaction between BuChE and nerve agents is diffusion limited, and that the BuChE concentration inside the vesicles is sufficiently high that nerve agent diffusion inside the polymerosome can be ignored:

$${C}_{polymerosome}^{critical} = \frac{1}{k} \cdot \frac{{r}_{free BuChE}^{2}}{{r}_{polymerosome}^{2}} \cdot {C}_{free BuChE}^{critical} ∼ \frac{1}{k} \cdot {10}^{- 6} \cdot {C}_{free BuChE}^{critical} ∼ k \cdot {10}^{- 14} \cdot mol \text{/} kg \qquad eqn 1 )$$

Here, r denotes radii of the polymerosome and the free BuChE, which are assumed to be in the order of 1 μm and 1 nm, respectively. k (with value between 0 and 1) is a probability factor that describes the likelihood that the nerve agent that collides with a polymerosome will diffuse through the membrane. This factor is influenced by a number of variables, including the surface density of pores on the polymerosome, the nerve agent diffusion rates in solution (3-D) and along the polymerosome surface (2-D). If the probability factor, k, is, conservatively, set to 0.01, the required concentration of circulating polymerosome is: C_{polymersome}^{critical}˜10⁻¹²·mol/kg.

If, also if is conservatively assumed that a circulation time of the polymerosomes is 10 days, the total number of polymerosomes needed is: n_{polymersome}^{normal}˜10⁻¹¹·mol/kg. That is, for performing mouse tests ˜10⁻¹³ mol˜10¹⁰ polymerosomes will be required per mouse.

Confining the BuChE within the polymerosome will increase the total number of BuChE proteins demanded for effective protection. If it is assumes that the concentration for BuChE inside the polymerosome is on the order of 1 mmol/L, each polymerosome will contain n=c·ν˜10⁻² mol/L·10⁻¹⁵ L˜10⁻¹⁹ mol˜10⁹ BuChE proteins. Correspondingly, the total concentration of BuChE will be:

$${C}_{BuChE in polymerosome}^{critical} = \frac{1}{k} \cdot \frac{{r}_{free BuChE}^{2}}{{r}_{polymerosome}^{2}} \cdot {C}_{free BuChE}^{critical} \cdot {10}^{9} ∼ \frac{1}{k} \cdot {10}^{3} \cdot {C}_{free BuChE}^{critical} \qquad eqn 2 )$$

However, extending circulation time is expected to compensate for this increased demand of BuChE.

The polymerosome capsules are fabricated using thin-shell double emulsions generated by applying bi-phasic flow capillary microfluidics, as pioneered by the Weitz team (Kim, Kim et al.). In this approach, highly monodisperse double emulsion drops are generated and subsequently converted into robust core-shell capsules, by consolidation of the ultra-thin middle layer (FIG. 2A). Compared to traditional approaches for making double emulsions, such as applying sequential inhomogeneous mechanical stirring, a much higher degree of control of capsule size, structure, chemical and mechanical properties, can be achieved using capillary microfluidics. Using a microfluidic approach, the shell thickness can be tuned by adjusting the relative flow rate of the middle phase fluid, adjusting the polymer/solvent ratio or by exploiting a co-flowing biphasic flow capillary geometry to form ultra-thin shells (Kim, Kim et al.); exploiting the thin shell technique enables us to form shells with thicknesses of 100 nm or less, which will facilitate the fast diffusion of toxins into the capsule core. The solidification of the drop middle phase can be done in three distinct ways; solvent evaporation (Lee and Weitz 2008), polymerization (Nie, Xu et al. 2005), or dewetting of the middle phase onto the surface of the innermost drop (Shum, Kim et al. 2008). Porous particles with precisely tuned pore size and density (Duncanson, Zieringer et al.; Carroll, Rathod et al. 2008) and capsules with porous membranes formed by activation of thermo-responsive polymers (Amstad, Kim et al.), have been made. These techniques are extended by applying liquid porogen templating and precipitation polymerization (Hao, Gong et al. 2009) of the drop middle phase to additionally tune the pore size of the resultant membranes. Importantly, the technique allows enzymes and other biomolecules to be encapsulated within biocompatible membrane materials including lipids and biodegradable polymers such as poly(lactic acid) (PLA). Here, particles of fully biodegradable materials, such as PLA with a controllable lifetime are fabricated.

Microfluidic devices based on flow focusing glass capillaries and polydimethylsiloxane (PDMS), have allowed the generation of double and higher order emulsion droplets with diameters of 60-100 μm (Utada, Lorenceau et al. 2005), (FIG. 2B-C) To allow formation of polymerosomes with diameters of 1-5 μm, devices with channels approaching these dimensions are fabricated. The flow rates and associated pressures required for droplet formation within such small channels require the devices to be based on more mechanically robust materials than glass and PDMS. Devices with channels as small as 2 μm are fabricated from, e.g., fluorinated polymers or stainless steel. using embossing techniques. Current devices allow droplet production at kHz frequencies. Consequently, drop production is scaled up by parallelizing drop making orifices; for example, by making a device with 100 parallel drop makers, it is possible to fabricate the 10¹⁰ capsules necessary for animal testing in less than 30 hours. Double emulsion drop production has been scaled up by parallelizing 15 drop making orifices on a single chip using soft lithography techniques (Romanowsky, Abate et al.).

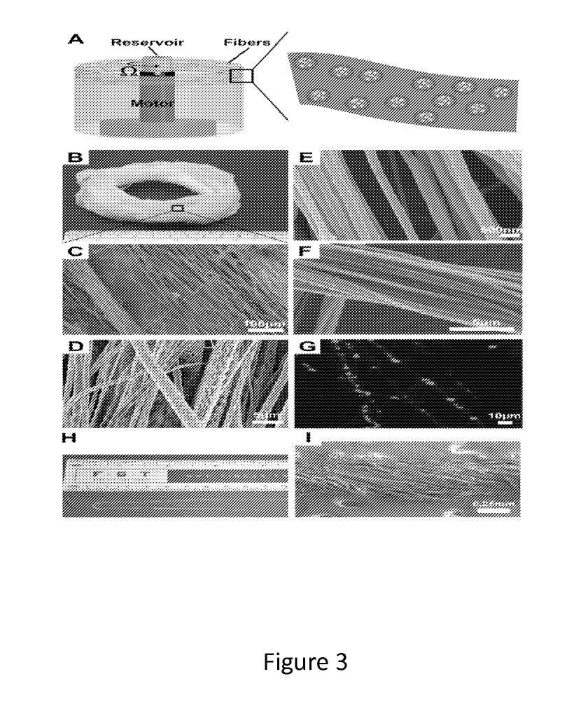

For fabricating biodegradable fibers and/or threads capable of delivering intact polymerosomes into circulation, Rotary Jet Spinning (RJS) is used. RJS is a micro- and nano-fiber production technique (Badrossamay, McIlwee et al. 2010). The technique utilizes centrifugal forces to extrude and elongate polymer jets from a reservoir rotating at up to 64,000 rpm through a 500 μm orifice, (FIG. 3A). Fibers have been made using various (bio)-molecules and solvents, including water. RJS is capable of producing nanofibers at 5-6× rate of electrospinning. The fabrication is performed at non-elevated temperatures and without applying electric fields that might destroy the molecular cargo, including BuChE (Badrossamay, McIlwee et al. 2010).

In order to control fiber degradation time and immune response, the ability to systematically vary fiber composition is central. In earlier reported studies, RJS has been used to produce fibers based Polyethylene glycol (PEG), polylactide (PLA), Poly(acrylic acid) (PAA), Gelatin, and composites thereof, see (FIG. 3). Particles have been encapsulated in the fiber, (FIG. 3G), and by varying the solution composition, tunable surface topographies (porous, beaded) have been made, (FIGS. 3D & F) (Badrossamay, McIlwee et al. 2010). For fabrication of polymeric fibers for use in the vehicles of the present invention, composites of biodegradable synthetic polymers such as Polycaprolactone (PCL) and poly(lactic-co-glycolic acid) (PLGA), and extracellular matrix (ECM) proteins such as Collagen (COL) and Fibronectin (FN) are used. Both slow and fast degrading sutures are of interest, dependent on the polymerosome circulation and lifetime. In addition to the composition, the size and mechanical properties of the fibers are regulated to allow suturing. It has been shown that the radius of fibers fabricated using RJS can be predicted by:

$$r ∼ \frac{{aU}^{1 / 2} {v}^{1 / 2}}{Ω {R}^{3 / 2}} \qquad eqn 3 )$$

Here r denotes fiber radius, R collector radius, ν kinematic viscosity of the solution, Ω angular speed, U the exit speed of the polymer jet from the reservoir, and a the initial jet radius (Mellado, McIlwee et al. 2011). Thus the fiber thickness is controlled a by varying external parameters such as rotational speed and solution viscosity. The United States Pharmacopoeia USP standard for sutures applied for wound closure is 40-600 μm in diameter with a tensile knot-pull strength of 1.38-62.3 N (Greenberg and Clark 2009). Because the objective is not wound closure, fibers with ultimate tensile strengths in the lower range of the USP standards and with stiffness approaching that of the subcutaneous tissue are fabricated.

### Fabrication of Polymerosomes, Biodegradable Polymeric Fibers and Threads, and Nanostructured Active Therapeutic Vehicles

Polymerosomes with selectively porous membranes are fabricated through in situ photopolymerization in the middle layer of a W/O/W double emulsion to form a consolidated cross-linked structure (FIG. 4). Biocompatible polymers such as polylactic acid (PLA), Poly(ε-caprolactone) (PCL), Polylactic acid co-glycolic acid (PLGA), Polyhydroxy butyrate (PHB), poly(ortho esters) (POE) and Poly-Hydroxybutyrate-co-b-Hydroxy valerate (PHBV) are covalently functionalized with acrylate and methacrylate groups using methacryloyl chloride to synthesize photo-polymerizable biodegradable polymers.

Established biphasic flow glass capillary devices are used to form W/O/W double emulsion template drops and ˜100 μm polymerosomes. The oil phase includes the synthesized photocurable polymers. The crosslinking density, and thus porosity is controlled by controlling the molecular weight and average number of acrylic functional groups on the polymeric chains. To reduce the UV exposure time needed for photopolymerization, a number of photoinitiators or combinations thereof, are included in the middle and outer phases. Fourier transform infrared spectroscopy (FTIR) is used to confirm the existence of covalent crosslinking bonds within the polymer backbone.

An alternative approach to controlling the porosity includes use of a porogen templating strategy. By dispersing the functionalized polymer in a non-reactive solvent which can also serve as porogen, upon UV exposure, precipitation polymerization occurs to form phase separated domains of crosslinked polymer and liquid porogen. Such a porogen solvent is non-halogenated so as not to hinder radical polymerization and has a low boiling point to facilitate selective removal after membrane consolidation. Suitable solvents include hexane, cyclohexane, 1,4-dioxane, ethers, and tetrahydrofuran. By controlling the ratio of dispersed polymer to porogen solution the shell thickness as well as membrane pore size is controlled. Low molecular weight liquid acrylic monomers or oligomers are also used which permit a much wider range of monomer to porogen ratio than is possible using large molecular weight precursors. For example, non-halogenated hydrocarbon oils with high boiling point are used to form these selective pores.

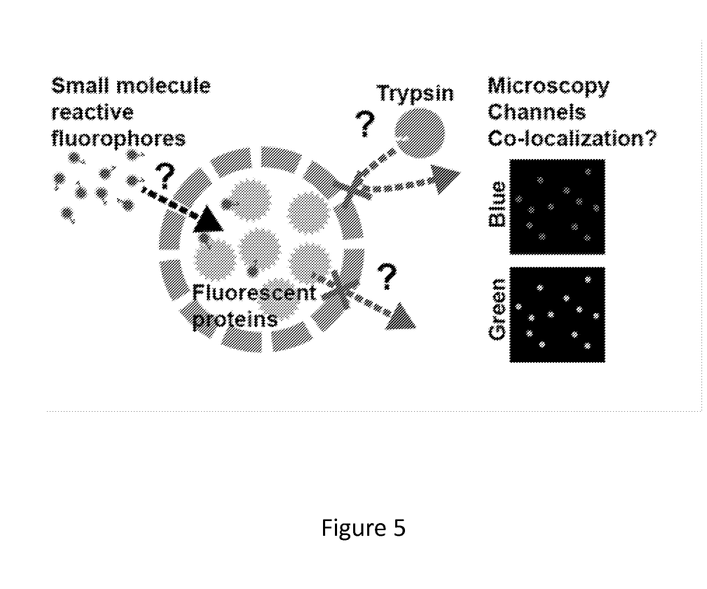

The selective permeability of the polymerosomes is determined in vitro by confocal microscopy (FIG. 5). By introducing fluorescently tagged proteins and inherently fluorescent proteins, to the internal water phase-polymerosome lumen, the ability of the fabricated polymersomes to encapsulate an active agent, such as BuChe, is evaluated. To ensure that the membranes, in addition to preventing leakage of the active agent, prevents degradation by proteases in the plasma, polymerosomes encapsulating labeled proteins are immersed in solutions containing proteases such as Trypsin and the ability of the polymerosomes to retain fluorescence is quantified. Chemically reactive fluorophores which bind covalently to proteins are used to mimic nerve agents binding to an active agent, such as BuChE. Upon entry to the polymerosome lumen such chemically reactive fluorophores bind irreversibly to the proteins and the fluorescence co-localize with that of e.g. GFP, see Table 1.

TABLE 1
Nerve Agents and reactive fluorophore phantoms

| | | Tert. | |
|---|---|---|---|
| | MW | Amine | Toxic |
| Nerve Agent | | | |
| Sarin | 140.09 | − | + |
| Tabun | 162.13 | + | + |
| VX | 267.37 | + | + |
| VR | 267.368 | + | + |
| EA-3148 | 279.378 | + | + |
| Fluorophore | | | |
| DACITC (7-Dimethylamino-4- | 260.31 | + | − |
| methylcoumarin-3-isothiocyanate) | | | |
| DNHS 7-Hydroxycoumarin-3- | 303.23 | − | − |
| carboxylic acid succinimidyl ester | | | |
| DACNHS (7-Diethylaminocoumarin- | 358.35 | + | − |
| 3-carboxylic acid succinimidyl ester) | | | |
| FITC (Fluorescein-5-isothiocyanate) | 389.382 | − | − |
| FNHS 5-(and 6-)carboxyfluorescein | 473.4 | − | − |
| succinimidyl ester | | | |
| RNHS 5-(and 6)- | 528 | + | − |
| carboxytetramethylrhodamine | | | |

“Dummy” particles are fabricated using single emulsion fabrication. An injection channel with 2 μm width is used to produce droplets in the range of 2-5 μm in diameter. Drops of 2-hydroxyethyl acrylate (HEA) or 2-hydroxyethyl mathacrylate (HEMA) monomer, photo initiator and poly(ethylene glycol) diacrylate (PEGDA) cross-linker are solidified using UV light. By using PEGDA with different molecular weights (1 kDa-24 kDa) and varying the cross-linker concentration from 10 wt % to 1 wt %, the elastic shear modulus of the hydrated polymeric particles is tuned to cover the range of the reported modulus for RBCs.

The porous particles are functionalized with amine groups by introducing 2-aminethyl acrylate and near-IR fluorescent dyes are covalently attach with NHS (N-hydroxysuccinimide) esters.

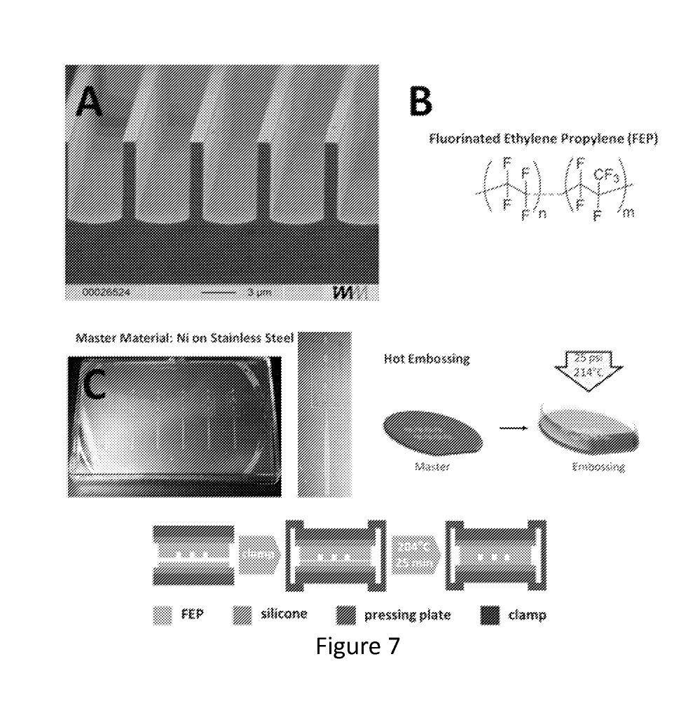

To fabricate hollow porous particle, such as polymerosomes, having a 1-5 μm diameter, the channels used for fabricating the double emulsion are reduced to similar dimensions. For emulsification of a small thread with dimensions of single microns, it is necessary to achieve large viscous shear stress; this makes droplet formation at these dimensions difficult as the associated pressures and flow rates will be large and mechanically robust materials are required. The pressure is approximated by the volumetric flow rate times the hydrodynamic resistance R for a square channel:

$$R = \frac{k η L}{{h}^{4}} \qquad eqn 4 )$$

where k=28.4 is a proportional constant for square channels, η is the fluid viscosity, L is the channel length, and h is the channel diameter. Because resistance scales as h−4, the pressure drop for single micron channels can be several MPa. Devices made from glass and PDMS materials cannot withstand pressures of this magnitude. Instead, devices with small microchannels are fabricated from mechanically robust materials such as stainless steel and Teflon; to make these devices, embossing techniques which facilitate fabrication of channels as small as 800 nm in diameter as illustrated in FIG. 7A are used (Becker, et al. 1998, Micro Total Analysis Systems '98, pp. 253-256).

A hot embossing fabrication method of microfluidic devices made of fluorinated polymers has been developed; the schematic describing this technique and images of the resultant devices are shown in FIG. 7C. These devices have been successfully employed to fabricate microparticles ranging in size from 2 μm to 100 μm. The fabrication process of perfluorinated microfluidic devices consists of three consecutive steps. As first step, the features are embossed in a commercially available Fluorinated Ethylene Propylene (FEP) sheet by hot embossing. Nickel electroplated on stainless steel sheets as a master may be used for the embossing. The pattern to be embossed is achieved by a photolithographic process; the resolution of the features is determined by the photo mask applied. With common photo masks, features down to 8 μm can be reliably obtained. Finer features are facilitated by the use of a chrome mask; these masks allow features as small as 2 μm. As second step, the FEP sheet containing the features is thermally bonded to another sheet at temperatures near the glass transition point of FEP. As third step, the surface properties of the channels are patterned. To render desired channel regions hydrophilic, these regions are flow patterned fusing a chemical etchant. The contact angle of water on FEP is decreased from 104° for untreated regions to approx. 35° for treated regions. The surface treatment of the channels allows the formation of double emulsion structures that depend on the spatially controlled wettability of channel walls.

The porosity of the miniaturized porous particles, e.g., polymerosomes, is characterized using an approach similar to that described above and, in addition, highly quantitative analysis of the porosity is performed. In particular, the capsules are freeze-dried to maintain the integrity of the membrane pores and gaseous physisorption analysis is used; details about the surface area and pore size distribution is obtained from measurements of the gas adsorption on the polymer surface as a function of temperature and pressure (Langmuir, 1918, Journal of the American Chemical Society, 40: 1361-1403). For determining pore size, a modified Kelvin equation (eqn 5) is used for non-complex pore structures. Alternatively or in addition, a non-localized density functional theory (NLDFT) method may be used for the case of hierarchical pore size (Carroll et al. 2009, Langmuir, 25(23):13540-4).

$$RT ln ( \frac{p}{{p}^{o}} ) = \frac{γ v}{r - {t}_{c}} \qquad eqn 5 )$$

where r is the radius of the cylindrical pore, p is the pressure of the gas, p^{o} is the condensation pressure, γ is the surface tension, ν is the molar volume of adsorbed gas, and t_{c} is the critical thickness of the adsorbate when capillary condensation will occur.

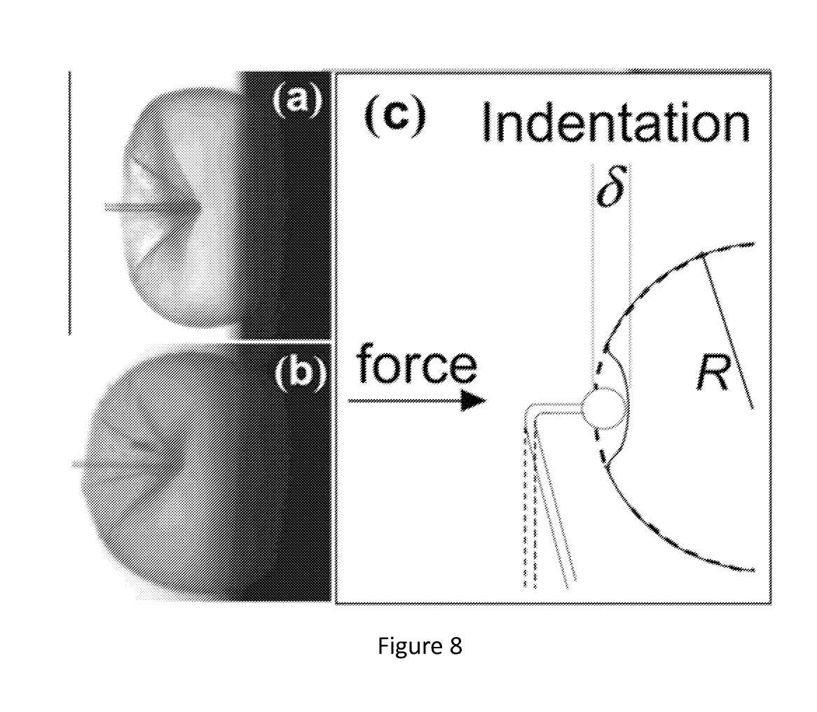

In addition to the methods for the synthesis of porous particles described above for fabricating 20-40 μm porous particles, e.g., polymerosomes, that are impermeable to molecules greater than 10 kDa in size, but permeable to molecules less than 500 Da, the elasticity of the capsules is tuned to ≦50 kPa to mimic that of red blood cells by synthesizing biodegradable latent acid polymers with diol co-precursors (see, e.g., Gordon et al. 2004, Journal of the American Chemical Society, 126(43): 14117-14122). Specifically, it has been shown that when indented by a microcantilever with a small hemispherical tip, essentially a point indenter, a deformed capsule conforms locally to the tip and elsewhere is convex with smoothly varying local curvature, as typified in (FIG. 8). From this linear response, a capsule spring constant in response to point indentation is estimated, from which a modulus for the capsule is defined. As an aid to inferring these capsules' structure from their mechanical response, finite element modeling is used to investigate the indentation of these spheres. For such a shell axisymmetrically deformed by a point load, dimensional analysis dictates that the indentation depth, ε, depends on the indentation force or load, P, and the initial internal pressure, p, as

$$\frac{δ}{R} = f ( \frac{P}{EtR} , \frac{PR}{{Et}^{3}} , \frac{pR}{2 Et} ) \qquad eqn 6 )$$

where t is the thickness, E the Young's modulus, and R is the radius of the shell. The first and second terms correspond, respectively, to the stretching and bending deformations caused by indentation. The third term is the nondimensionalized internal pressure. A shell's effective stretching stiffness is Et/(1−ν²) and its effective bending stiffness is Et³/12(1−ν²), where ν is Poisson's ratio; the bending stiffness depends more strongly on the shell thickness than does the stretching stiffness. Capsules are deformed using calibrated microcantilevers and finite element modeling is used to measure the capsules' mechanical response. For capsules approaching the dimensions of red blood cells, a small colloid attached to an atomic force microscopy (AFM) cantilever is used.

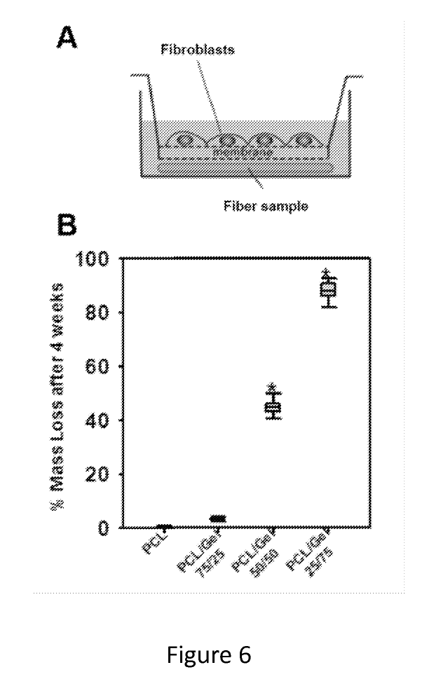

Combining different ratios of biodegradable block co-polymers provides an effective method for controlling the degradation rates of the membrane polymer. For instance 90:10 poly([rac-lactide]-co-[ε-caprolactone]) degrades in 2 months. By increasing the ratio of one polymeric block, such as PCL, this degradation time is increased to one year. Alternatively, or in addition, degradation rates are tuned by synthesizing biodegradable latent acid polymers using different ratios of diol and ether lactide precursors; this synthesis approach provides precise control of alpha hydroxyl acid segments in the polymer that controls the erosion rate. Erosion rates are determined in vitro by exposing the polymer to an aqueous solution; the degradation products of the polymer are isolated from the solution and characterized. Initially, degradation is accelerated to achieve faster characterization results by performing these tests at elevated temperature (70° C.) and alkaline pH. The resultant degraded products are injected into HPLC or GPC columns for precise molecular weight characterization of the oligomers. The porous particle, e.g., polymerosome, degradation is determined in an in vitro cellular environment using methods similar to that applied for the polymeric fibers and/or threads (described below) and, illustrated in (FIG. 6A).

To facilitate long circulation times in the blood stream and inhibit phagocytosis of the capsules, the polymers are modified with different functional moieties such as carboxyl or amine groups and PEG and/or inhibitory bio molecules such as CD47 are attached to the capsule surface using various coupling reactions. A procedure similar to that outlined for the N-IR labeling of the “dummy” particles described above may be used. In particular, amine groups are introduced in the particles using amine-reactive compounds, such as NHS ester methyl-capped PEG. As an alternative approach, PEG functionalized with acrylic groups may be dispersed in the aqueous continuous fluid and linked to the surface of the polymer containing only acrylic groups during in-situ photopolymerization (FIG. 4).

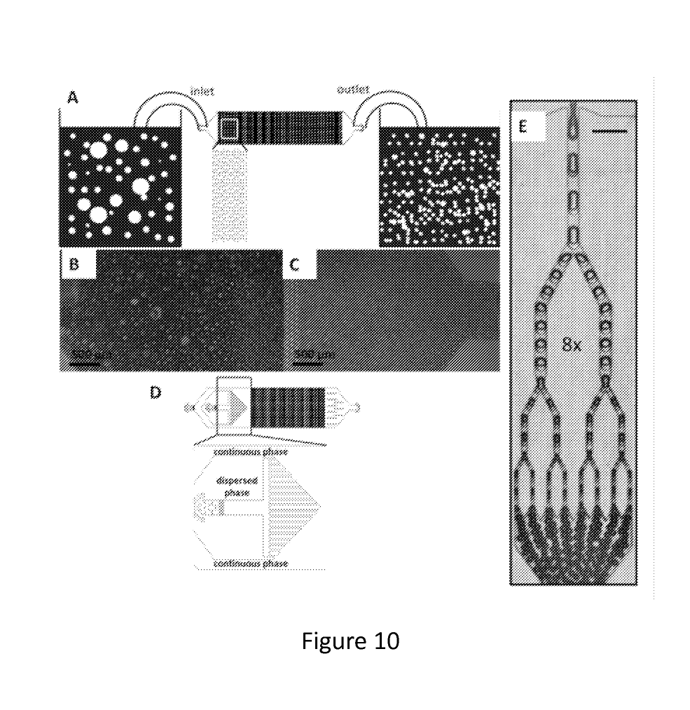

To scale up capsule production of porous particles for in vivo testing, a parallel numbering-up design for microfluidic double emulsification devices is used (Romanowsky et al. 2012, Lab on a Chip, 12(4): 802-807; see, e.g., FIG. 10). This technique increases throughput greatly while maintaining good product uniformity. The basic dropmaker units are repeated in both a two-dimensional and a three-dimensional array, and are connected using a three dimensional network of much larger distribution and collection channels. Up to 100 dropmaker units are integrated to produce single-core double emulsion drops at rates of 100,000 drops per second, equivalent to 10¹⁰ capsules in 30 hours which provides the number of porous particles necessary for animal testing.

As an alternative strategy for high throughput production of template drops, a microfluidic filter that allows high through-put production of emulsions may be used. This approach employs a device consisting of a single inlet where an emulsion, produced through bulk emulsification, is injected; the emulsion is sheared by the microfilters which consist of posts that are arranged in rows with well-defined distances. This produces significantly smaller drops that have a narrower size distribution than the injected bulk drops. The device schematic and processed drops are shown in (FIG. 10).

A variation of the microfluidic device involves on-chip formation of large drops; subsequently, these large drops are broken up into smaller more monodisperse drops as they are forced through the arrays as shown in FIG. 10. Using this version of the filters permits the production of double emulsions on-chip shortly before the emulsion drops are further broken up into smaller drops. The applicability of these devices to high-throughput formation of double emulsions is achieved by tuning the geometry and spacing of the post junctions (see, e.g., Abate and Weitz, 2011, Lab on a Chip, 11(11): 1911-1915). As the drops encounter a junction, the lobes lengthen, eventually remaining connected by only a narrow coaxial thread; as the thread narrows the outer interface squeezes on the inner drop, narrowing it, and causing it to eventually snap, dividing the double emulsion drop into two, as shown in FIG. 10. These double emulsions are split into even smaller drops by the next two forks in similar processes.

Biodegradable polymeric fibers and/or threads are fabricated using Rotary Jet Spinning Devices (RJS) by combining FDA approved biodegradable polyesters, such as PCL and PGLA, and ECM proteins to produce fibers and/or threads with controlled degradation time, facile release of embedded porous particles, e.g., polymerosomes, and good tissue integration. By adjusting the ratio of polylactic acid (PLA) and polyglycolic acid (PGA), the degradation time of PLGA co-polymers is finely tuned from 1-2 months (50:50 PGA:PLA) to 6-8 months (15:85 PGA:PLA) (Ulery et al., 2011, Journal of Polymer Science Part B-Polymer Physics, 49(12): 832-864). As an alternative to PLGA, PCL is used (Dash, T. K. and V. B. Konkimalla, 2012, Journal of Controlled Release, 158(1): 15-33; Dash, T. K. and V. B. Konkimalla, 2012, Molecular Pharmaceutics, 9(9): 2365-2379).

The chemical composition of the fibers and/or threads is characterized using ATR-FTIR spectroscopy and SEM imaging is used to determine fiber and/or thread structure and thickness. An Instron 3345 with a 1 kN load cell is used to determine the stiffness and ultimate tensile strength of the fibers and/or threads. To determine the fiber and/or thread degradation time, an in vitro assay as outlined in FIG. 6A is used. Briefly, human fibroblasts are seeded in transwell membrane plates within 6-well plates containing fibers and/or threads. Fiber and/or thread samples are collected throughout a 3 month period of continuous culturing of the cells; the fibers are dried and weighed, their composition investigated using ATR-FTIR, and imaged using SEM. In addition to adding Fibronectin and Collagen, more soluble proteins may be used to fabricate the fibers and/or threads to further control the degradation time and particle release characteristics of the fibers and/or threads. Highly soluble proteins such as gelatin and albumin, or glycosaminoglycans such as hyaluronic acid, are used for this purpose. FIG. 6B shows that the degradation of PCL-Gelatin composite fibers and/or threads is dependent on the Gelatin content (FIG. 6B). To assess the biocompatibility of the fibers and/or threads, fibroblasts are cultured directly on substrates of the fabricated fibers and/or threads. At selected time points during a 3-month period, cells are fixed and immune-stained with nuclear and nucleus cytoskeletal markers to assess cell condition.

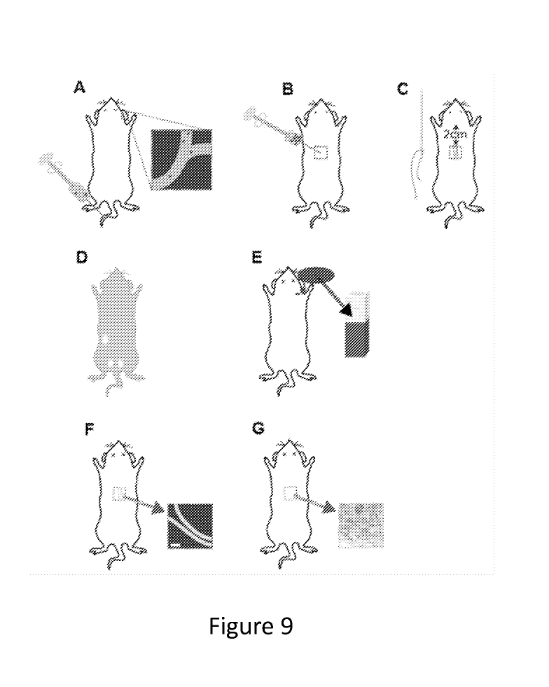

In vivo analysis of the degradation, immune response and delivery capabilities of the fabricated fibers and/or threads are performed. For example, N-IR labeled dummy microparticles and fiber compositions are used. Three types of studies, each defined by a different administration method, are performed. In particular, free particles are injected intravenously (FIG. 9A), free particles are injected subcutaneously, (FIG. 9B) and a biodegradable polymeric fibers and/or threads comprising porous particles are delivered subcutaneously through a suture (FIG. 9C). To demonstrate circulation of the particles in mouse vessels, intravital microscopy is used (FIG. 10A) (Merkel, et al., 2011, PNAS, 108(2): 586-591). N-IR fluorescence of the whole mouse under anesthesia using a IVIS live Infrared Imaging system is used to show potential aggregation of the microparticles in specific tissue, (FIG. 9D). Organs collected after sacrifice of the mouse are also imaged to demonstrate that the particles do not aggregate. For determining the amount of microparticles entering circulation, trunk blood or blood collected via cardiac puncture is assessed for fluorescence in the N-IR range, (FIG. 9E), and analyzed for the presence of fluorescent particles using FACS. For the studies using fibers and/or threads to deliver the particles, animals under inhaled anesthetic are shaved and prepped for aseptic surgery and a thread of degradable fibers encapsulating microparticles, is introduced subcutaneously approximately 2 cm caudal to the scapulae, (FIG. 9C). Alternatively, to allow larger amounts of fiber to be introduced in one area, a small subcutaneous pocket is created cephalically, the fiber sample inserted, and the pocket sealed. A number of additional evaluations are performed on sacrificed animals with implanted sutures: To determine the degradation speed of the implanted fibers, the remaining fibers are collected, rinsed, weighed and the fiber diameter evaluated using conventional microscopy and SEM, (FIG. 9F). To identify potential immunologic responses histology of the insertion sites is performed, (FIG. 9G). Hematoxylin & Eosin stain along with immunospecific stains, such as CD68 labeling of macrophages, are used. Histology of the insertion sites is also used to assess fiber degradation and microparticle release. These studies are summarized in Table 2.

TABLE 2
In vivo Analysis of Delivery of Dummy Porous Particles and Biodegradable
Polymeric Fibers and/or Threads Comprising a Dummy Porous Particle.

| | Minimal | | Intravi- | | Blood N-IR | | |
|---|---|---|---|---|---|---|---|
| | No. of | | tal Mi- | IVIS N-IR | emission | His- | Fiber Size |
| Study | Mice | Sacrifice time | croscopy | Imaging | & FACS | tology | Evaluation |
| IV injected dummy particles | 5 | 3 hrs | X | X | X | | |
| Subcutanous free | 8 × 5 | 1 day, 3 days, 7 days | | X | X | X | |
| dummy particles | | 30 days, 90 days. | | | | | |
| Fiber delivery of | 2 × 8 × 5 | 1 day, 3 days, 7 days | | X | X | X | X |
| dummy particles | | 30 days, 90 days. | | | | | |

Similar analyses are used for the in vivo analysis of a nanostructured active therapeutic vehicle as described herein (see, e.g., Table 3).

TABLE 3
Mice studies of delivery of selectively permeable polymerosomes through a degradable suture

| | | | Intravi- | | Blood N-IR | | |
|---|---|---|---|---|---|---|---|
| | No. of | | tal Mi- | IVIS N-IR | emission | His- | Fiber Size |
| Study | Mice | Sacrifice time | croscopy | Imaging | & FACS | tology | Evaluation |
| IV injected polymerosomes | 5 | 3 hrs | X | X | X | | |
| Subcutanous free | 8 × 5 | 1 day, 3 days, 7 days | | X | X | X | |
| polymerosomes | | 30 days, 90 days. | | | | | |
| Fiber delivery of | 2 × 8 × 5 | 1 day, 3 days, 7 days | | X | X | X | X |
| polymerosome | | 30 days, 90 days. | | | | | |

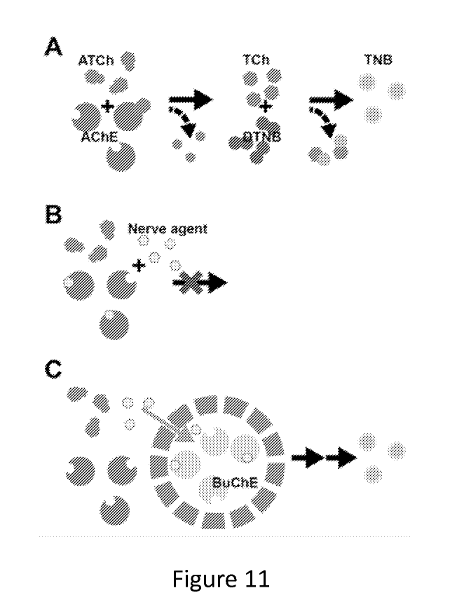

The ability of porous particles, e.g., polymerosomes, encapsulating BuChE to capture nerve agents is initially assessed in vitro. In particular, in addition to acetylcholine, Acethylcholine esterase activity (AChE) hydrolyzes a number of other choline esters including the thioester acetylthiocholine (ATCh) to thiocholine (TCh). This is the basis of the Ellman assay, in which the activity of AChE is estimated by measuring the absorbance of thiobisnitrobenzoate (TNB) formed by reaction between TCh and dithiobisnitrobenzoate (DTNB) (FIG. 11A). When a nerve agent binds to AChE it becomes inactive (FIG. 11B), and consequently, the ability of polymerosomes encapsualting BuCE to inhibit the inactivation of AChE by nerve agents, can be assessed by the ability of the polymerosomes to restore the formation of the colored TNB product of the assay (FIG. 11C). The in vitro tests are performed in buffer and/or blood samples. The absorption (X) of TNB overlaps with that of hemoglobin, so in order to perform tests in blood, DTNB is replaced by an alternative chromophore precursor such as 2,2′-dithiodipyridine (2-PDS). (Miao, et al., 2010, Chemical Reviews, 110(9): 5216-5234). This assay verifies the activity of the polymerosomes using the mild acethylcholine esteraseinhibitor Diethyl Fluoro Phosphate (DFP) as nerve agent, due to the restriction on use of more potent nerve agents such as sarin and VX. Porous particles are removed from solution by dialysis prior to assaying Acethylcholine esterase activity.

The ability of the nanostructured active therapeutic vehicles of the invention to counteract nerve agents in vivo is performed by subcutaneously administering vehicles comprising BuChE as sutures on the dorsal side of mice and/or rats. At predetermined time intervals the mice are exposed to a nerve agent. Repetitive seizures indicate lethality: all animals exhibiting clonic/tonic seizures for more than 2 minutes are immediately euthanatized via an overdose of 200 mg/kg pentobarbital.

Additional in vivo assays are performed as outlined in Table 4 to determine the single and multiple doses of IV-injected DFP that provides i) 50% inhibition of plasma cholinesterase; ii) observable but mild muscle fasciculation in 60 to 80% of the mice dosed; and iii) repetitive muscle fasciculation in 60 to 80% of the mice dosed.

TABLE 1
in vivo nerve agent protection studies on mice

| | | No. Of | |
|---|---|---|---|
| Acute Mouse Study 1 | DFP dose | Mice | dose label |
| non-sutured mice | increased until 50% | 30 | “low” |
| (control) | plasma choline esterase | | |
| non-sutured mice | increased until mild | 20 | “moderate” |
| (control) | siezure observed | | |
| non-sutured mice | increased until chronic | 30 | “high” |
| (control) | seizure >2 mins | | |

| | | No. Of | Exposure and sacrifice |
|---|---|---|---|
| Acute Mouse Study 2 | DFP dose | Mice | time points (days) |
| polymerosome sutured mice | high | 8 × 5 | 3, 7, 14, 30, 90 |
| polymerosome sutured mice | moderate | 8 × 5 | 3, 7, 14, 30, 90 |
| polymerosome sutured mice | low | 8 × 5 | 3, 7, 14, 30, 90 |
| non-sutured mice (control) | high | 8 × 5 | 3, 7, 14, 30, 90 |
| non-sutured mice (control) | moderate | 8 × 5 | 3, 7, 14, 30, 90 |
| non-sutured mice (control) | low | 8 × 5 | 3, 7, 14, 30, 90 |

| | | No. Of | Sequential exposure |
|---|---|---|---|
| Acute Mouse Study 3 | DFP dose | Mice | points (days) |
| polymerosome sutured mice | high | 20 | 7, 14, 30, 90 |
| polymerosome sutured mice | moderate | 20 | 7, 14, 30, 90 |
| polymerosome sutured mice | low | 20 | 7, 14, 30, 90 |
| non-sutured mice (control) | high | 20 | 7, 14, 30, 90 |
| non-sutured mice (control) | moderate | 20 | 7, 14, 30, 90 |
| non-sutured mice (control) | low | 20 | 7, 14, 30, 90 |

### EQUIVALENTS

In describing exemplary embodiments, specific terminology is used for the sake of clarity. For purposes of description, each specific term is intended to at least include all technical and functional equivalents that operate in a similar manner to accomplish a similar purpose. Additionally, in some instances where a particular exemplary embodiment includes a plurality of system elements or method steps, those elements or steps may be replaced with a single element or step. Likewise, a single element or step may be replaced with a plurality of elements or steps that serve the same purpose. Further, where parameters for various properties are specified herein for exemplary embodiments, those parameters may be adjusted up or down by 1/20th, 1/10th, ⅕th, ⅓rd, ½, etc., or by rounded-off approximations thereof, unless otherwise specified. Moreover, while exemplary embodiments have been shown and described with references to particular embodiments thereof, those of ordinary skill in the art will understand that various substitutions and alterations in form and details may be made therein without departing from the scope of the invention. Further still, other aspects, functions and advantages are also within the scope of the invention.

Exemplary flowcharts are provided herein for illustrative purposes and are non-limiting examples of methods. One of ordinary skill in the art will recognize that exemplary methods may include more or fewer steps than those illustrated in the exemplary flowcharts, and that the steps in the exemplary flowcharts may be performed in a different order than shown.

### INCORPORATION BY REFERENCE

The contents of all references, including patents and patent applications, cited throughout this application are hereby incorporated herein by reference in their entirety. The appropriate components and methods of those references may be selected for the invention and embodiments thereof. Still further, the components and methods identified in the Background section are integral to this disclosure and can be used in conjunction with or substituted for components and methods described elsewhere in the disclosure within the scope of the invention.

1. A nanostructured active therapeutic vehicle, comprising
a biodegradable polymeric fiber comprising a porous particle or a biodegradable polymeric thread comprising a porous particle, wherein the porous particle comprises regulators that control passage of molecules into and out of the particle, and wherein the porous particle comprises an active agent.

2. A nanostructured active therapeutic vehicle for sustained delivery of an active agent, comprising a biodegradable polymeric fiber or a biodegradable polymeric thread and a polymerosome comprising the active agent, wherein the active agent is an agent which inhibits the activity of a toxin, and wherein the polymerosome comprises size regulators which control passage of molecules into and out of the particle such that the active agent is excluded from exiting the polymerosome, a molecule which degrades the active agent is excluded from entry into the polymerosome, and the toxin is permitted entry into the polymerosome such that the toxin contacts the active agent, thereby inhibiting the activity of the toxin.

3. The nanostructured active therapeutic vehicle of claim 1 or 2, wherein the biodegradable polymeric fiber or biodegradable polymeric thread comprises synthetic and/or natural polymers.

4. (canceled)

5. (canceled)

6. The nanostructured active therapeutic vehicle of claim 1 or 2, wherein the polymeric fiber or biodegradable polymeric thread is about 1 to about 1,000 micrometers in diameter or about 10 to about 100 micrometers in diameter.

7. (canceled)

8. The nanostructured active therapeutic vehicle of claim 1 or 2, wherein the polymeric fiber or biodegradable polymeric thread has a tensile strength of about 0.5 N to about 100 N or about 1 N to about 50 N.

9. (canceled)

10. The nanostructured active therapeutic vehicle of claim 1, wherein the porous particle is selected from the group consisting of an emulsion product, a microgel, a particle whose pores may be templated by micelles, microemulsion drops, dendrimers, colloids, liquid porogen, lipids, degree of polymeric crosslinks, a dendrimer, a micelle and combinations thereof.

11. The nanostructured active therapeutic vehicle of claim 10, wherein the emulsion product is a polymerosome.

12. The nanostructured active therapeutic vehicle of claim 2, wherein the polymerosome has a diameter of about 0.1 to about 10 micrometers or a diameter of about 0.5 to about 5 micrometers.

13. (canceled)

14. The nanostructured active therapeutic vehicle of claim 2, wherein the polymerosome has a shell with a thickness of about 50 to about 500 nanometers.

15. The nanostructured active therapeutic vehicle of claim 2, wherein the polymerosome is impermeable to molecules greater than about 10 kiloDaltons, but permeable to molecules about 5 to about 500 Daltons.

16. The nanostructured active therapeutic vehicle of claim 2, wherein the polymerosome has a stiffness of about 5 to about 100 kiloPascals.

17. The nanostructured active therapeutic vehicle of claim 2, wherein a middle layer of the polymerosome comprises a polymer selected from the group consisting of poly(ε-caprolactone), PLA, PLGA, PHB, POE, PHBV, copolymers, and derivatives thereof.

18. The nanostructured active therapeutic vehicle of claim 2, wherein an outer layer of the polymerosome further comprises polyethylene glycol or CD47.

19. The nanostructured active therapeutic vehicle of claim 1 or 2, wherein the active agent is selected from the group consisting of small molecules, nucleic acid based drugs; polypeptides; peptides; proteins; carbohydrates; polysaccharides and other sugars; glycoproteins, and lipids.

20. The nanostructured active therapeutic vehicle of claim 1 or 2, wherein the active agent is butyrlcholinesterase.

21. The nanostructured active therapeutic vehicle of claim 1 which provides release of the active agent for about 1 week to about 1 month or about 1 week to about 3 months.

22.-44. (canceled)

45. A method for providing sustained release of an active agent to a subject having a condition treatable with the active agent, comprising administering to the subject an effective amount of a nanostructured active therapeutic vehicle comprising the active agent, wherein the nanostructured active therapeutic vehicle comprises a biodegradable polymeric fiber comprising a porous particle or a biodegradable polymeric thread comprising a porous particle, wherein the porous particle comprises regulators that control passage of molecules into and out of the particle, and wherein the porous particle comprises an active agent, thereby providing sustained release of the active agent to the subject having a condition treatable with the active agent.

46. A method for providing sustained release of an active agent which inhibits the activity of a toxin in a subject, comprising administering to the subject an effective amount of a nanostructured active therapeutic vehicle comprising an active agent that inhibits the activity of the toxin, wherein the nanostructured active therapeutic vehicle comprises a biodegradable polymeric fiber comprising a polymerosome or a biodegradable polymeric thread comprising a polymerosome, and wherein the polymerosome comprises size regulators which control passage of molecules into and out of the particle such that the active agent is excluded from exiting the polymerosome, a molecule which degrades the active agent is excluded from entry into the polymerosome, and the toxin is permitted entry into the polymerosome such that the toxin contacts the active agent, thereby providing sustained release of an active agent which inhibits the activity of a toxin to the subject.

47. The method of claim 46, wherein the subject is at risk of being exposed to the toxin.

48. A method for inhibiting the activity of a toxin in a cell, comprising contacting the cell with a nanostructured active therapeutic vehicle comprising an active agent capable of inhibiting the activity of the toxin, wherein the nanostructured active therapeutic vehicle comprises a biodegradable polymeric fiber comprising a porous particle or a biodegradable polymeric thread comprising a porous particle, wherein the porous particle comprises regulators that control passage of molecules into and out of the particle, and wherein the porous particle comprises an active agent, thereby inhibiting the activity of a toxin in the cell.

49. The method of any one of claims 45-48, wherein the active agent is selected from the group consisting of small molecules, nucleic acid based drugs; polypeptides; peptides; proteins; carbohydrates; polysaccharides and other sugars; glycoproteins, and lipids.

50. The method of any one of claims 45-48, wherein the active agent is butyrlcholinesterase.

51. The method of any one of claims 45-48, wherein the nanostructured active therapeutic vehicle comprising an active agent is administered to the subject subcutaneously.

52. The method of claim 51, wherein the subcutaneous administration comprises subcutaneous suturing.

53.-100. (canceled)
